(19) United States
(12) Patent Application Publication
(10) Pub. No.: US 2025/0186003 A1
(43) Pub. Date: Jun. 12, 2025

(54) DEVICE, SYSTEM AND METHOD FOR BED EXIT MONITORING

(71) Applicant: ASA Robotics Limited, Kowloon (HK)

(72) Inventor: Wah Shing LAM, Kowloon (HK)

(21) Appl. No.: 18/972,417

(22) Filed: Dec. 6, 2024

Related U.S. Application Data
(60) Provisional application No. 63/606,768, filed on Dec. 6, 2023.

(51) Int. Cl. A61B 5/00; A61B 5/11

(52) U.S. Cl. CPC A61B 5/746; A61B 5/1115; A61B 5/7405; A61B 2560/0252

(57) ABSTRACT

The application discloses a system, device and method for bed exit monitoring. The system includes: a distance sensor array comprising distance sensors and a sensor array module, each distance sensor operable to measure the distance between the human and the sensor, and output corresponding distance signal; a sensor array module operable to receive and process the distance signals output by each distance sensor and output a distance signal matrix according to all the distance signals; a thermal sensor configured to measure a temperature in a range of the bed and output a corresponding temperature signal; a processing unit, configured to process a distance signal matrix and a temperature signal to determine a human body position, and output a position state signal of the human body; a first alarm unit, configured to identify the position state signal output by the processing unit and make corresponding feedback.

### CROSS-REFERENCE TO RELATED APPLICATIONS

This application claims the benefit of U.S. provisional patent application No. 63/606,768, filed on Dec. 6, 2023, which is incorporated herein by reference in its entirety.

### TECHNICAL FIELD

The present invention relates to movement monitoring and in particular to systems, devices and methods for bed exit monitoring.

### BACKGROUND

In clinical medicine, there are patients who are not suitable to get up, but some patients may not follow the doctor's advice and leave the bed without permission, which will bring adverse effects on the later treatment. In addition, some elderly people in part of nursing and home care are also not suitable for getting out of bed. Therefore, it is very necessary to monitor the bed-exiting behavior of some patients and old people in real time.

Known bed exit monitoring systems include those relying on infrared fence, such as Safe Concept Infrared Bed-exit Detector, these systems use two types of infrared sensors, human motion sensor and distance detection sensor. The infrared beams will detect the body movement and position of the user. Such systems can be prone to false alarms if the beam is blocked for innocent reasons, such as movements of a visitor or nursing staff in the vicinity of the bed.

Other bed exit monitoring systems rely on pressure mat, such as Medicus Tek Sensable Care System. Medicus Tek is a solution for both fall prevention and pressure ulcer prevention. The technology is based on a multi-point smart sensor in the form of a bed sheet on top of a normal mattress or supplied as a hospital mattress with embedded sensors. Body pressure on sensors create a pressure “heat map” with read out of number of hours for each point. Tracking body positions, pressure and duration makes life easier for caregiver and patient well being. Reminders can be set in smart devices to alert caregiver to attend to reposition a patient. It is the only system that approaches ideal case of real fall prevention instead of commonly found systems as fall alert after a patient had fallen off bed. Based on tracked behavior caregiver smart device can be set to level of alertness thus predicting if someone is about to leave bed or just sitting up or shifting sitting positions. Pressure sensors in the form of mats require constant maintenance, including cleaning, disinfection and repositioning before usage, causing an extra workload for caregivers. Furthermore, most of these pressure sensors feature audible alarm functions, which could cause distress to other residents in hostels, especially elderly people with dementia. It is therefore crucial to minimize unnecessary alarms caused by false alarms but at the same time give vocal feedback to patients in order to help them understand the alarm and wait for an assistant from caregiver.

Another type of bed exit monitoring systems rely on lidar, such as Family AI Fall Detection Lidar System. Family AI Fall Detection Lidar System includes: lidar sensor, detecting object distance by reflection and AI computing. When the system detects a fall or an user without any movement in a certain period, an alert will be instantly sent out. When an user stays too long in the covering area, a reminder will be sent out. Lidar device suggested installing in the ceiling, recommend with 2.5 m headroom or above. Processor can install in the ceiling or any location near the lidar device, requires power with 40 W, 220V. Alarm can be installed in any conspicuous places. This kind of system makes intelligent analysis based on the collected data, which has high complexity and large amount of calculation, leading to high cost and difficult to popularize and apply in a wide range.

Still other bed exit monitoring systems rely on millimeter wave technology, such as Care Tech System Limited-Millimeter wave radar fall detection system. Using millimeter-wave radar (mmWave) electromagnetic wave reflection signals to detect the distance, speed and angle of objects, collecting 4D point cloud information. Combined with a self-developed AI deep learning engine, it performs human pose recognition to determine if a fall has occurred and instantly notifies caregivers.

There are many other types of systems, such as Monibed Infrared Bed-exit Monitor User Manual with thermal array, and Altum View Sentinare with Colour camera, etc. There are one or more technical problems with the above system: frequent false alarm; such as failing to classify an actual action/movement of a person; limitation for installation; weakness in terms of privacy; difficulties in understanding the meaning of alarm.

### SUMMARY

The present invention provides a system, device and method for bed exit monitoring that at least partially overcomes the above problems.

According to a first aspect of the present disclosure, there is provided a bed exit monitoring system, comprising: a distance sensor array comprising distance sensors and a sensor array module, wherein each distance sensor operable to measure the distance between the human and the sensor in different angles, and output corresponding distance signal; a sensor array module operable to receive and process the distance signals output by each distance sensor and output a distance signal matrix according to all the distance signals; a thermal sensor configured to measure a temperature in a range of the bed and output a corresponding temperature signal; a processing unit, configured to process a distance signal matrix of the distance sensor array and a temperature signal of the thermal sensor to determine a human body position, and output a position state signal of the human body; a first alarm unit, configured to identify the position state signal output by the processing unit and make corresponding feedback.

In an embodiment, the distance sensors have different detection angles, and the detection angles of the distance sensor array covers the entire bed and the surrounding area of the bed.

In an embodiment, the distance sensor may be a Time of Fight lidar sensor (ToF lidar sensor).

In an embodiment, the bed exit monitoring system further includes a temperature sensor, wherein the temperature sensor communicates with the processing unit, and is configured to measure a room temperature and output a corresponding temperature signal.

In an embodiment, a classification module is arranged in the processing unit, and programmed to classify a human body position into safe, abnormal, or high-risk states based on the distance signal matrix of the distance sensor array and the temperature signal output by the thermal sensor and the temperature sensor.

In an embodiment, the bed exit monitoring system further includes a remote device, the remote device includes a second alarm unit, the second alarm unit is configured to identify a position state signal output by the processing unit and make a corresponding feedback, and the second alarm unit may generate an auditory alarm and/or a visual alarm.

In an embodiment, the remote device further includes a display unit, and the display unit communicates with the processing unit and is configured to present a real-time human body position state.

In an embodiment, the bed exit monitoring system further includes a communication module, and the communication module is configured to transmit a position state signal of the human body.

In an embodiment, the bed exit monitoring system further includes a cloud server, the processing unit uploads the position state signal to the cloud server, and the cloud server sends the position state signal to the remote device.

According to a second aspect of the present disclosure, a bed exit monitoring device is provided, including a case, a distance sensor array, a thermal sensor, a print circuit board assembly (PCBA), and a speaker assembly; a chamber is disposed inside the case, and a switch is disposed outside the case; the distance sensor array and the thermal sensor are disposed on the case and face the bed body; the distance sensor array includes distance sensors and a sensor array module; the PCBA is disposed in the chamber of the case and includes a processor, and the processor is in communication with the distance sensor array and the thermal sensor; and the speaker assembly is disposed on the case and in communication with the processor.

In an embodiment, the bed exit monitoring device further includes a bracket disposed on the bed frame, and the case is disposed on the bracket.

In an embodiment, the case is configured as a cat model, including a front case and a rear case, and the front case and the rear case are detachably connected. The speaker assembly includes a speaker and a volume button, the speaker is disposed on the front case, and the volume button is disposed on the rear case.

In an embodiment, the bed exit monitoring device further includes a temperature sensor disposed on the rear case and configured to measure the room temperature.

In an embodiment, the bed exit monitoring device further includes a power supply.

In an embodiment, the bed exit monitoring device further includes a remote device, and the remote device may be in communication with the processor.

In an embodiment, the bed exit monitoring device further includes a cloud server, and the cloud server may be in communication with the processor and the remote device.

According to a third aspect of the present disclosure, there is provided a bed exit monitoring method, comprising:
installing the bed exit monitoring device on the bed frame;
witching on the device and connecting to a cloud server;
measuring, by the distance sensor array, the distance between human body and the distance sensor array; and measuring, by the thermal sensor, the temperature within the bed body; and measuring, by the temperature sensor, the room temperature;
determining, by the processor, a human body position based on the spatial distance readings from the sensor array and also the temperature difference between the thermal sensor reading and the temperature sensor reading, and determining which human body position state the human in;
uploading, by the processor, the human body position states to a cloud server; sending, by the cloud server, the human body position states to the remote device;
displaying, by the remote device, a human body position state, and feeding back corresponding prompt information in different states.

In an embodiment, the step of measuring comprises: each distance sensor in the distance sensor array measures a distance between a human body and a sensor in different angles, and outputs a corresponding distance signal; a sensor array module of the distance sensor array receives and processes the distance signal output by each distance sensor, and outputs a distance signal matrix according to all the distance signals. The thermal sensor is configured to measure a temperature in a range of the bed body, and output a corresponding temperature signal. The temperature sensor is configured to measure the room temperature, and output a corresponding temperature signal.

In an embodiment, the distance sensor array's measurement range further comprises the area around the bed, allowing the processor to determine whether there is a caregiver around the bed, and when there is a caregiver, not triggering an alarm.

Furthermore, wherein the determining which human body position state the human in comprises: when the results of the distance sensor array readings and the temperature difference between the thermal sensor reading and the temperature sensor reading meet a first preset condition, determining, by the processor, the position of the human body as a safe state; when the above results meet a second preset condition, determining, by the processor, the human body position as an abnormal state; when the above results meet a third preset condition, determining, by the processor, the human body position as a high-risk state; when the above results meet the fourth preset condition, determining, by the processor, that a caregiver exists around the bed body.

Optionally, the first preset condition to the fourth preset condition are set according to a spatial size of the detection range.

Techniques of the present application can provide substantial beneficial technical effects. By way of example only and without limitation, according to one or more embodiments of the application may provide one or more of the following advantages.

The first point, the disclosure equipped with TOF Lidar sensors and a thermal sensor allows continuous and accurate monitoring of patient movement and actions. This enables healthcare providers to have real-time insights into patient behavior, enhancing patient safety and care.

The second point, the classification module within the processor can identify about normal patient actions, such as a tempting to leave or move the bed's edges, based on sensor data. This early detection enables timely interventions to prevent falls or other hazardous situations, reducing the risk of patient injuries.

The third point, the vocal voice prompting mechanism of the disclosure can deliver customized messages based on the detected abnormal patient actions. This personalized approach helps to effectively communicate with patients and remind them to avoid potentially risky movements, promoting patient compliance and safety.

The fourth point, the remote device receives alerts and alarms when the system detects a patient leaving the bed. This immediate notification allows nursing staff to promptly respond and provide assistance, reducing the likelihood of patient accidents or wandering.

The fifth point, the use of multiple TOF Lidar sensors positioned radially outward provides a comprehensive view of the patient's surroundings. This allows accurate spatial mapping obstacle detection, and avoidance, enhancing the navigation capabilities in the patient's environment.

The sixth point, the automated monitoring and intervention capabilities of the disclosure relieve healthcare staff of constant vigilance, allowing them to focus on other critical tasks. Simultaneously, patients benefit from a sense of security and receive timely reminders and assistance when needed, leading to an improved overall healthcare experience.

The seventh point, the present disclosure alters elderly action with vocal voice prompting feedback.

The eighth point, the present disclosure has a low false alarm rate.

The ninth point, the present disclosure plugs and plays on bed.

The tenth point, the present disclosure may preserve patient privacy.

The eleventh point, the present disclosure may classify different posture with different scenarios.

The twelfth point, the present disclosure has multiple installation in a room.

These and other features and advantages of the present invention will become apparent from the following detailed description of illustrative embodiments thereof, which is to be read in connection with the accompanying drawings.

### BRIEF DESCRIPTION OF DRAWINGS

Non-limiting and non-exhaustive embodiments of the present invention will be described with reference to the following drawings which are presented by way of example only, wherein like reference numerals (when used) indicate corresponding elements throughout the several views unless otherwise specified, and wherein:

FIG. 1 is a perspective view of the bed exit monitoring device in one possible embodiment.

FIG. 2 is an installation diagram of the bed exit monitoring device in one possible embodiment.

FIG. 3 is a exploded view of the bed exit monitoring device other than the bracket in one possible embodiment.

FIG. 4 is a front view of the bed exit monitoring device other than the bracket in one possible embodiment.

FIG. 5 is a rear view of the bed exit monitoring device other than the bracket in one possible embodiment.

FIG. 6 is a schematic diagram of the measurement angles of the distance sensor array in one possible embodiment.

FIG. 7 is a block diagram of the bed exit monitoring system in one possible embodiment.

In the drawings: 21—case, 22—distance sensor array, 23—thermal sensor, 24—circuit board assembly, 25—speaker assembly, 26—bracket, 27—temperature sensor.

It is to be appreciated that elements in the figures are illustrated for simplicity and clarity. Common but well-understood elements that may be useful or necessary in a commercially feasible embodiment may not be shown in order to facilitate a less hindered view of the illustrated embodiments.

### DETAILED DESCRIPTION

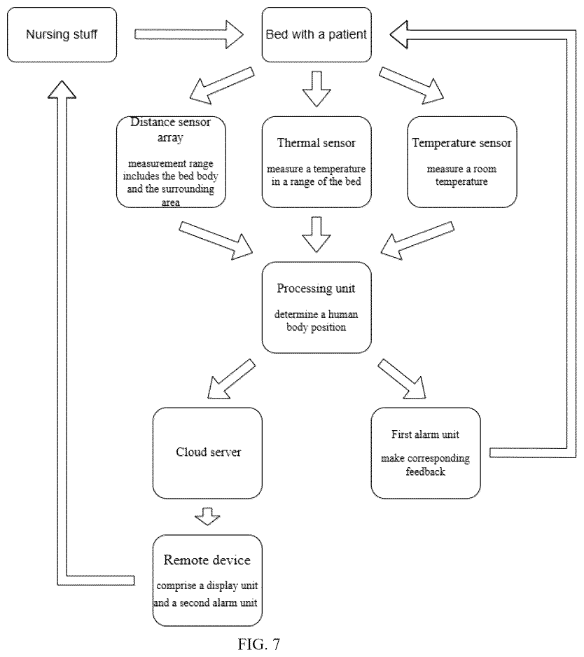

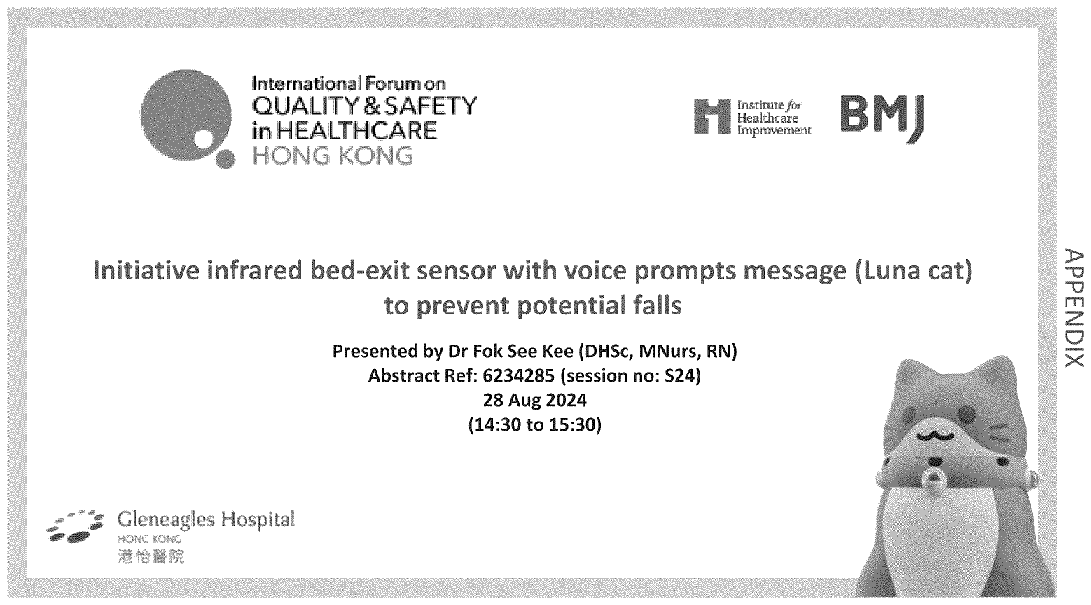

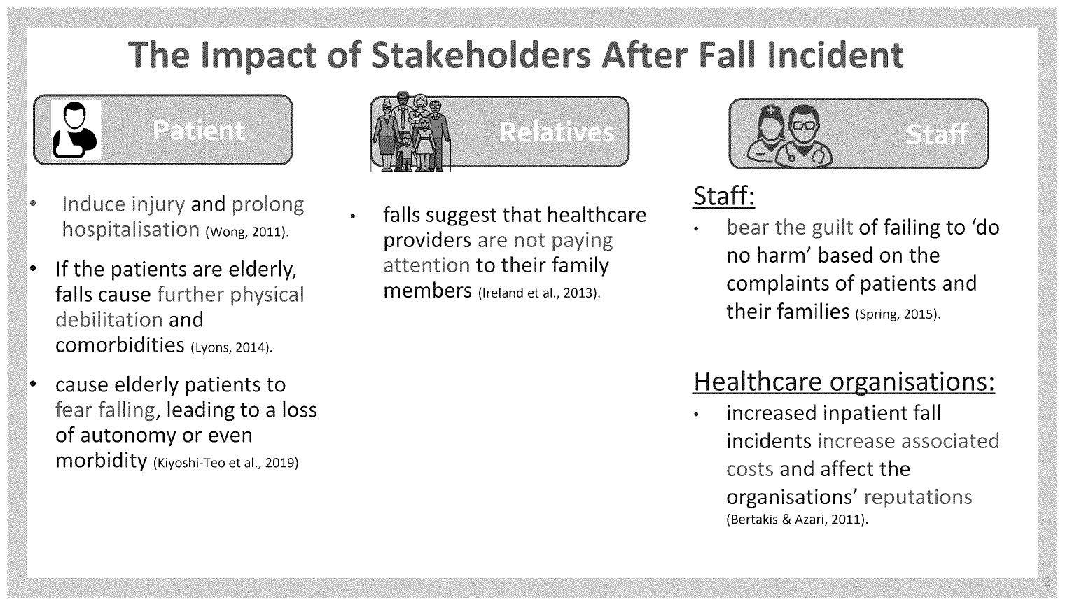

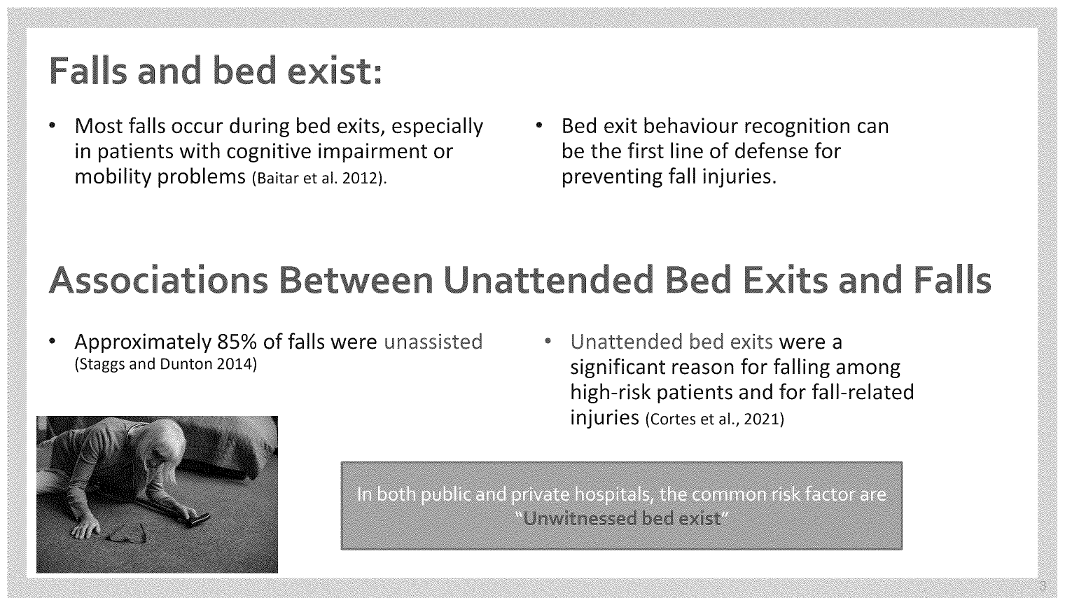

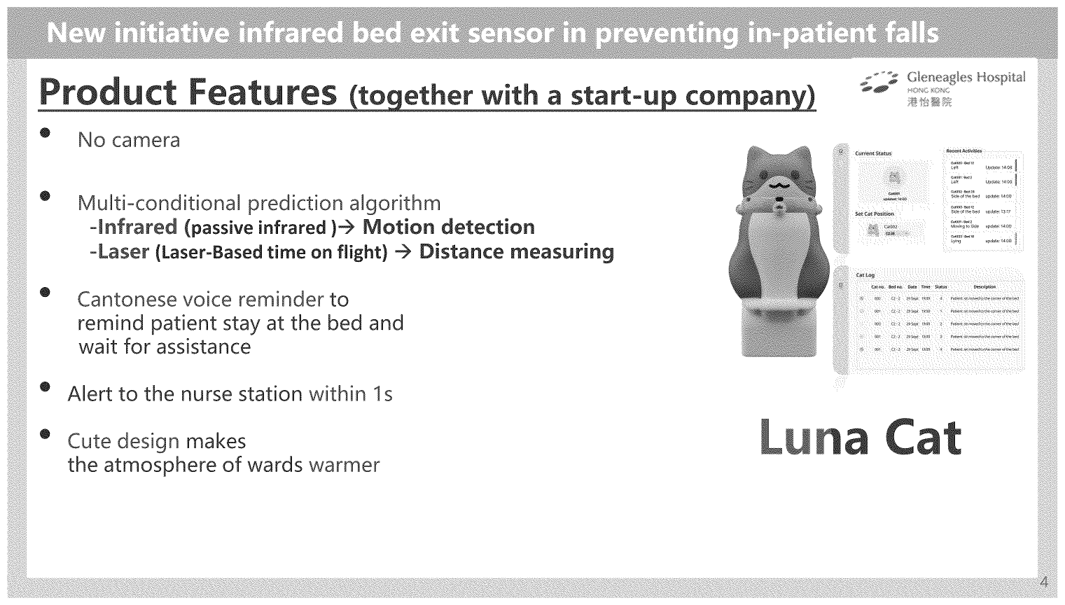

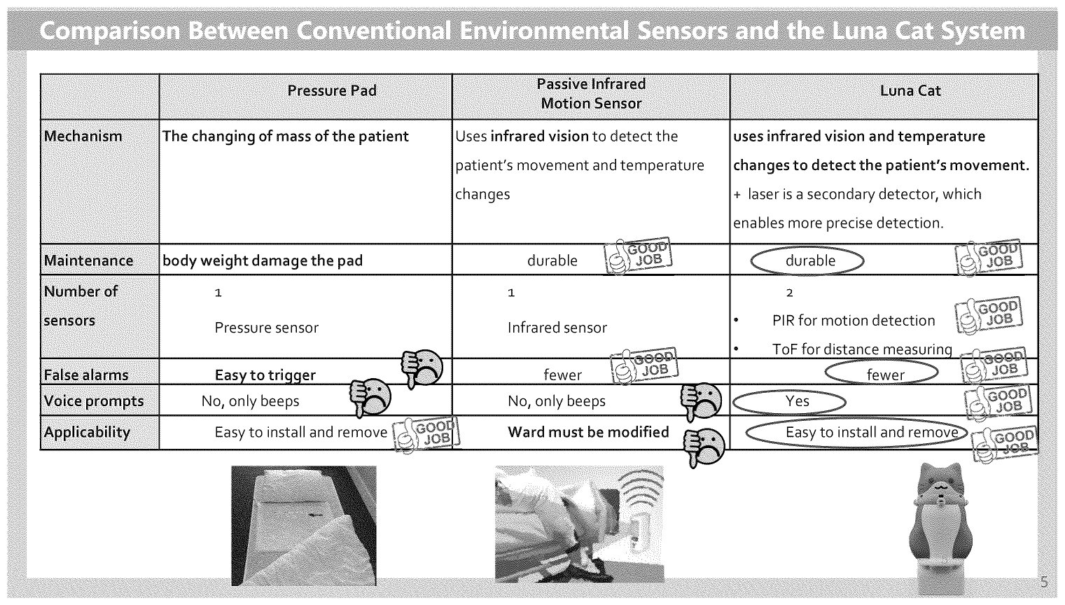

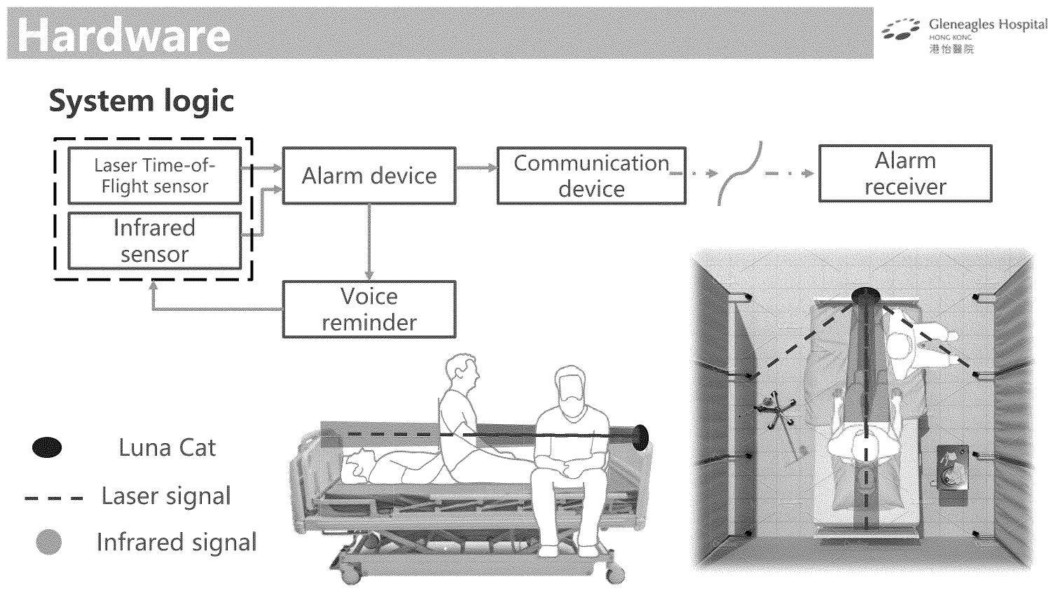

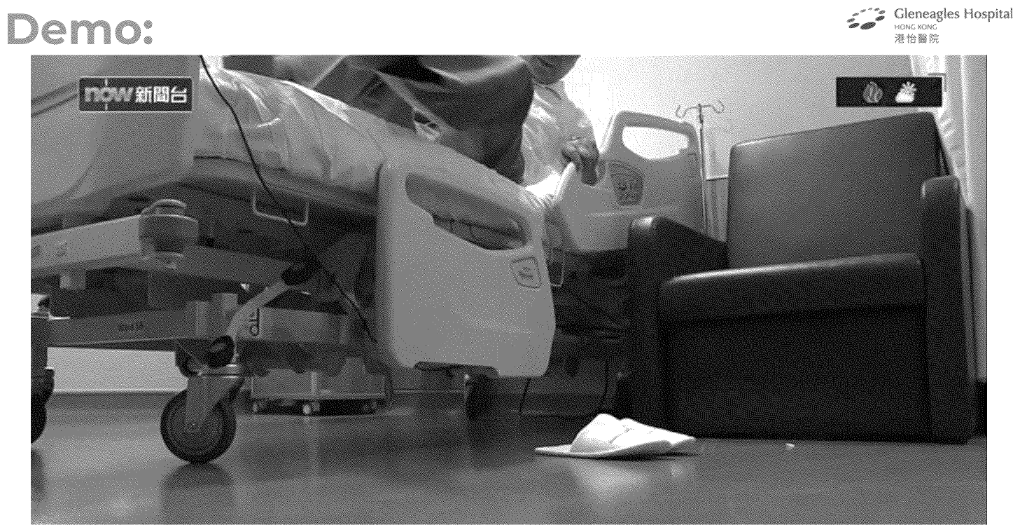

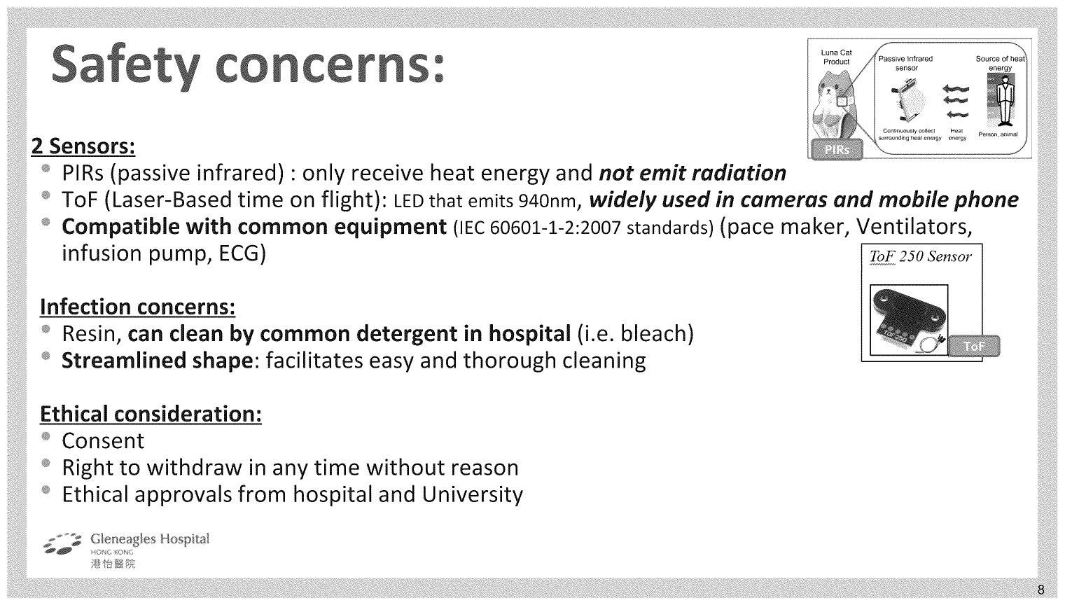

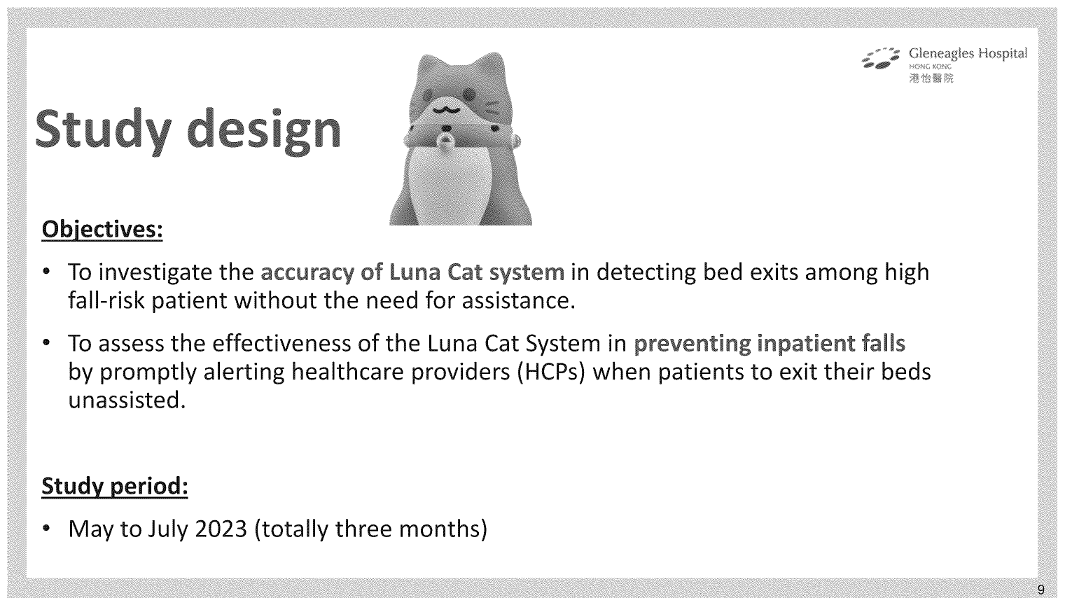

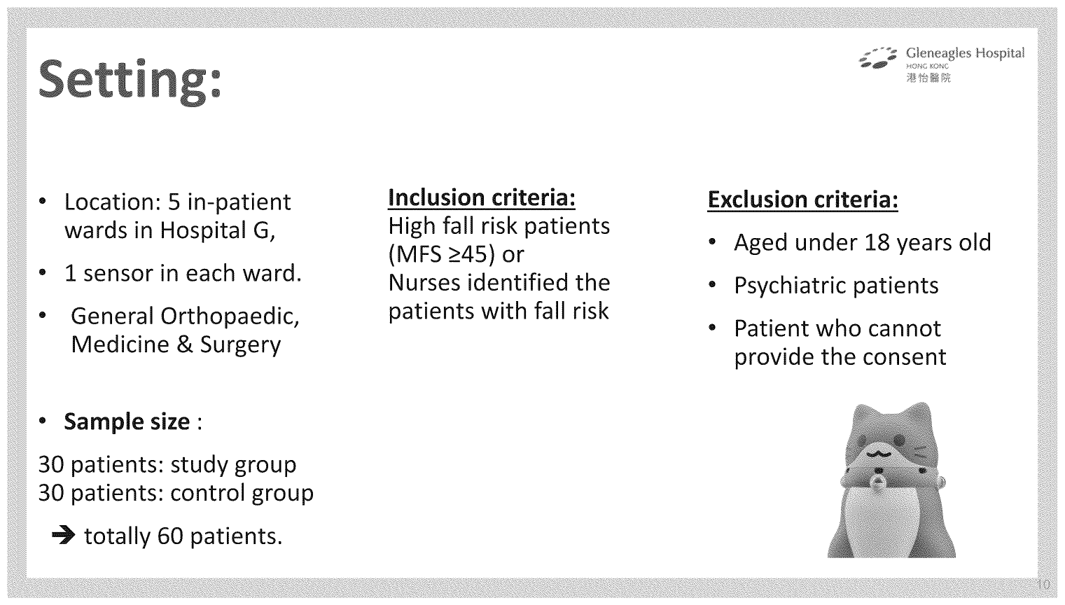

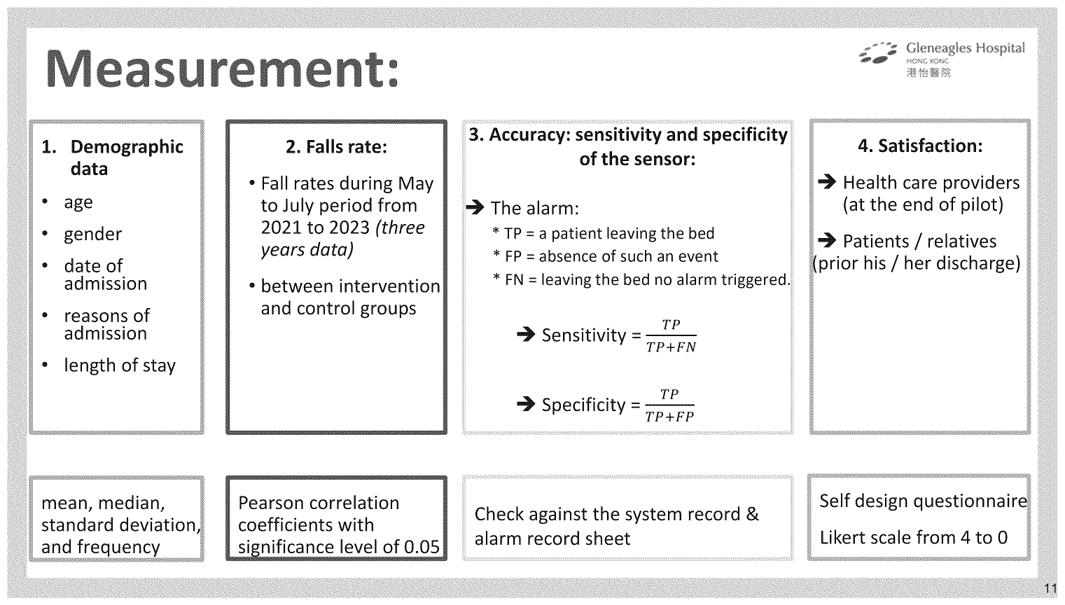

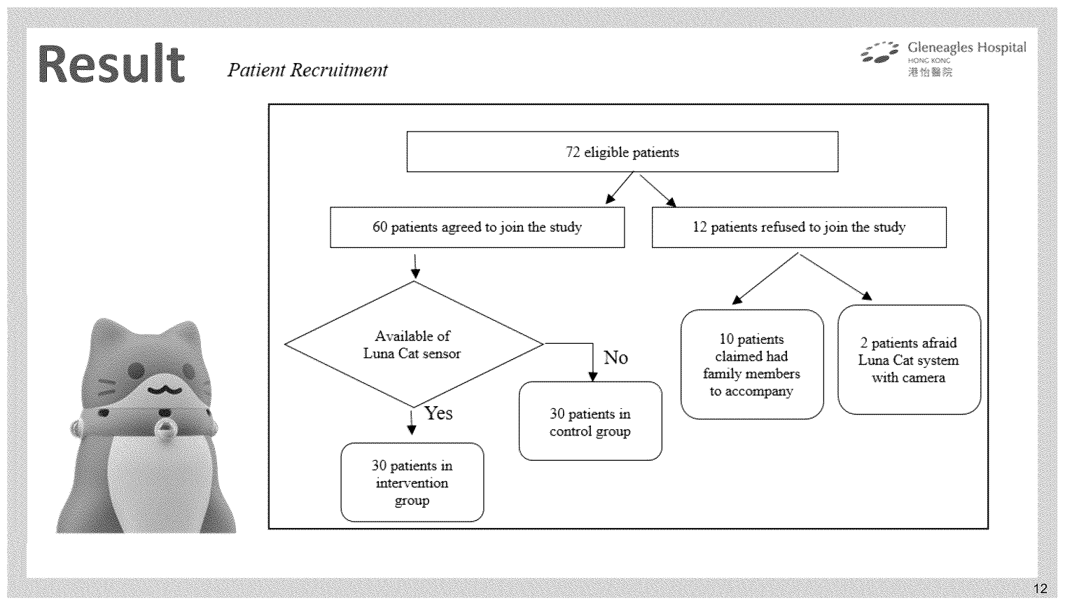

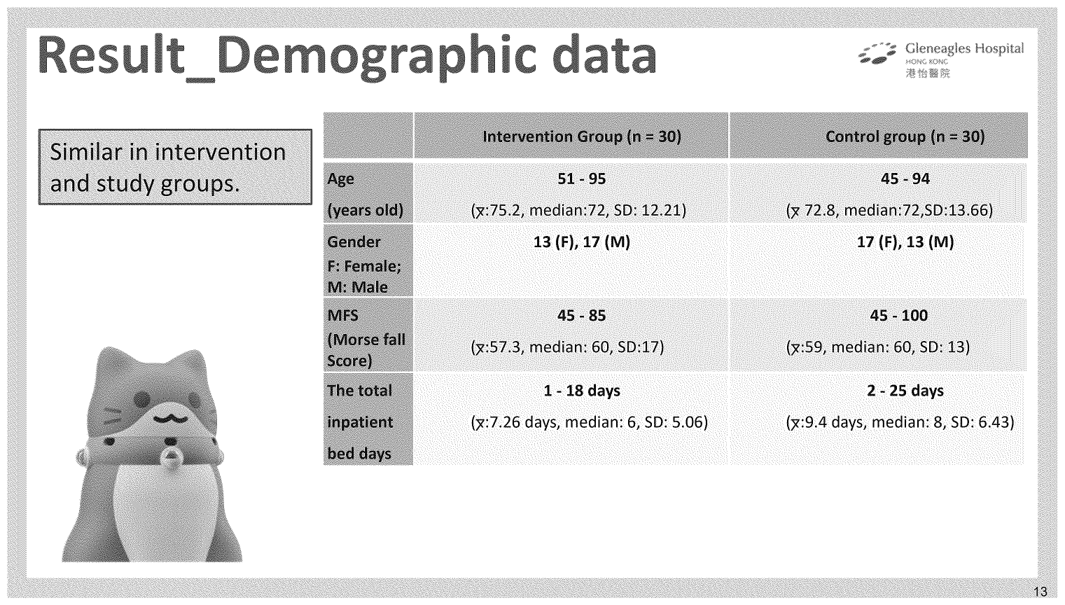

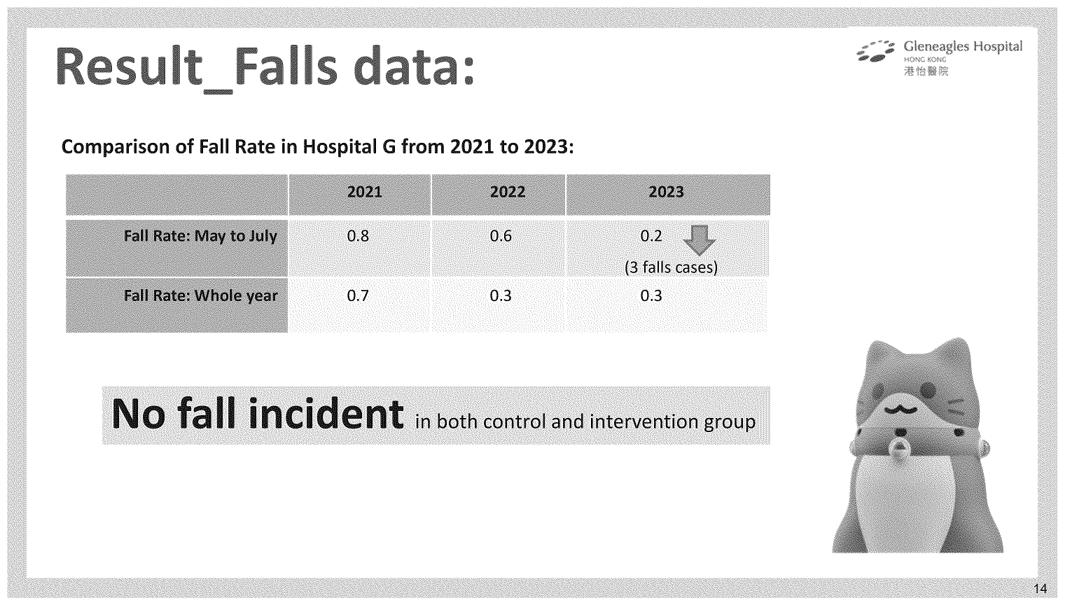

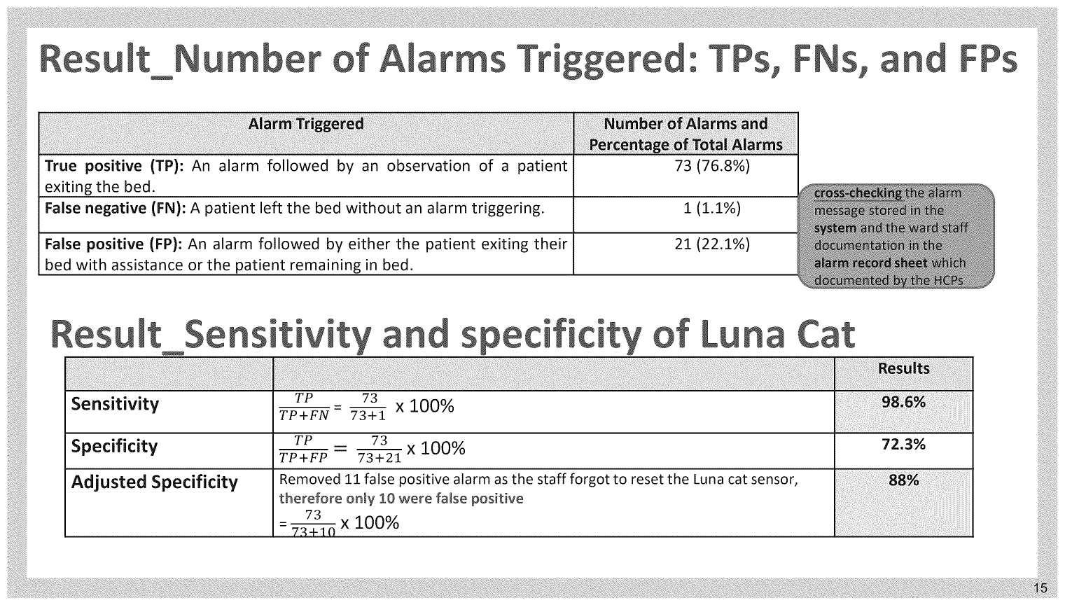

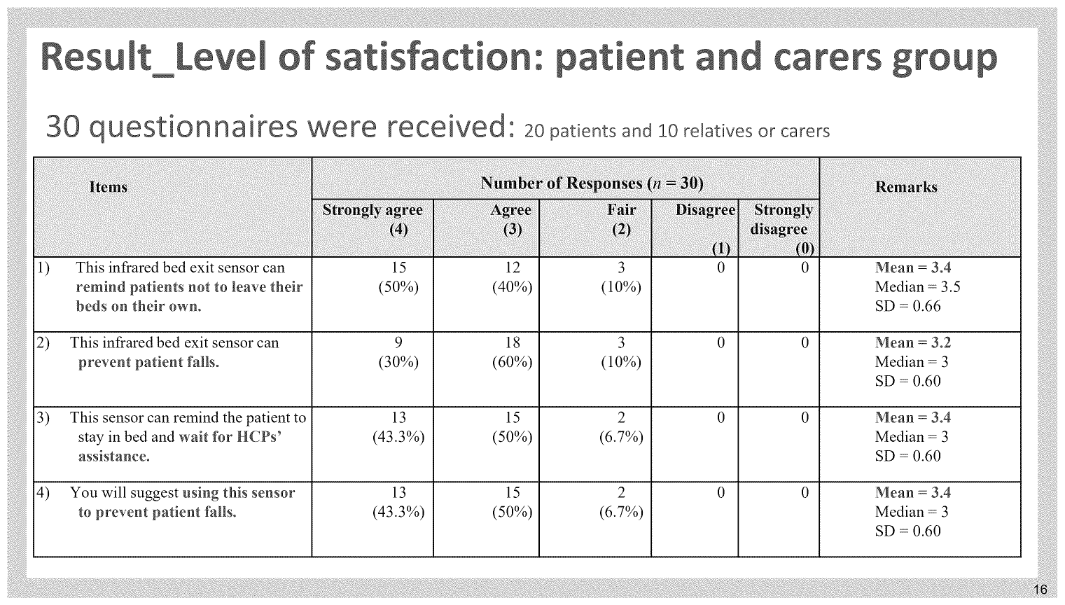

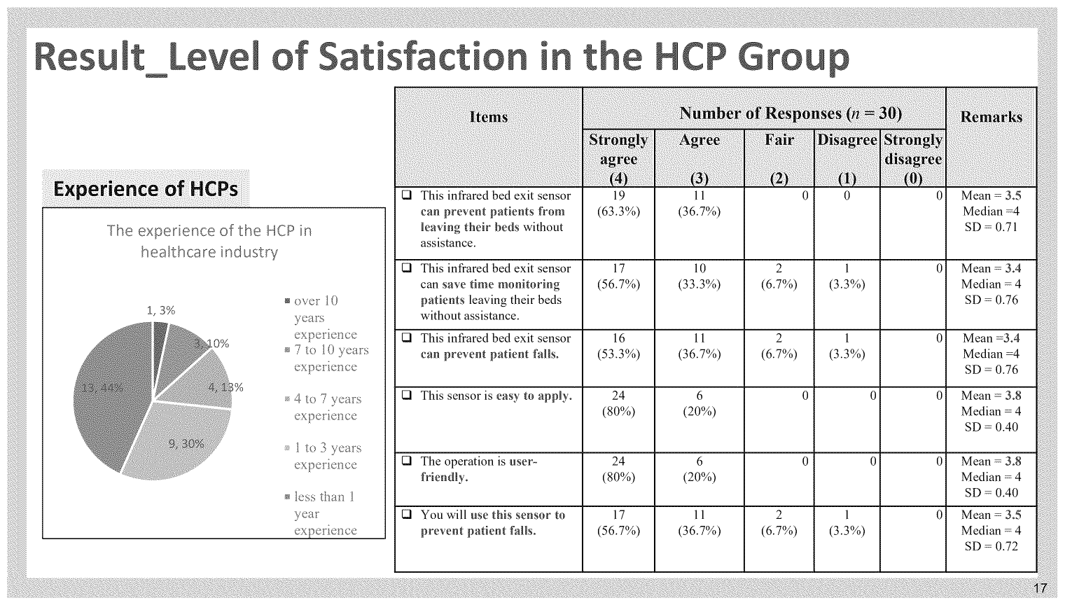

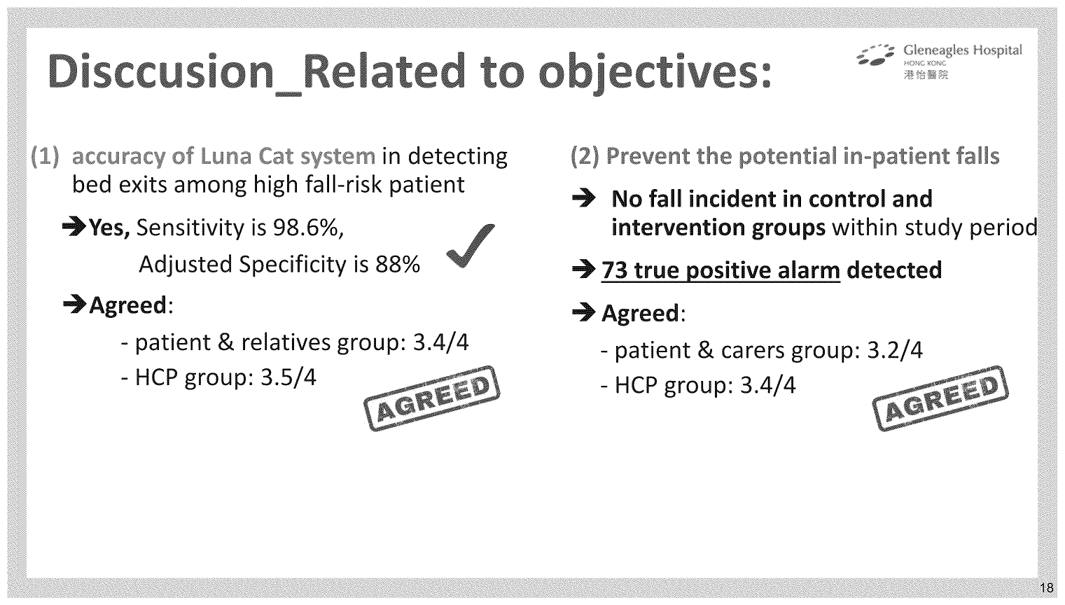

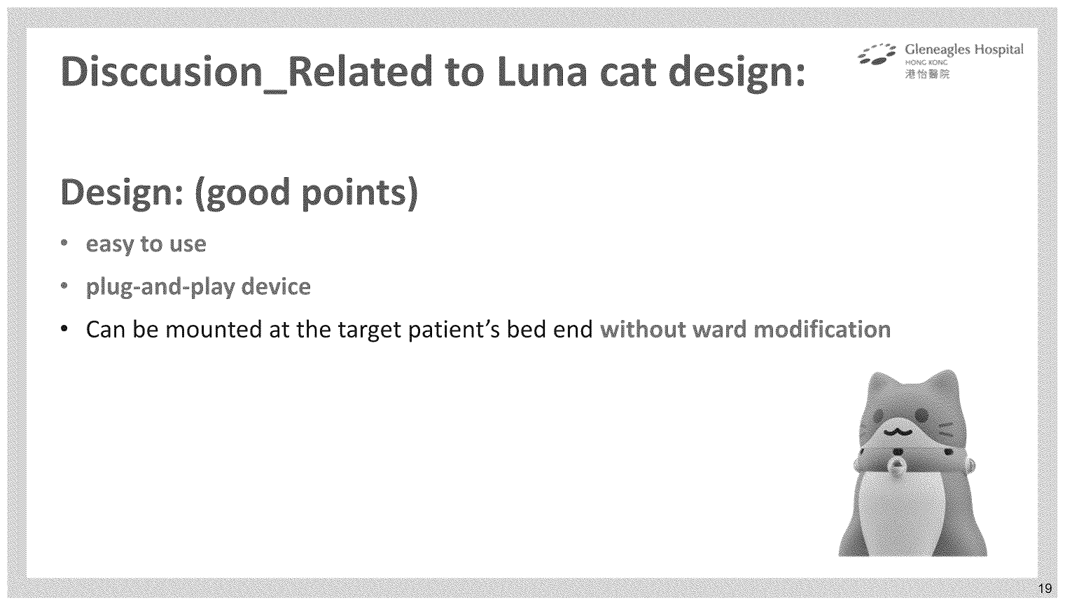

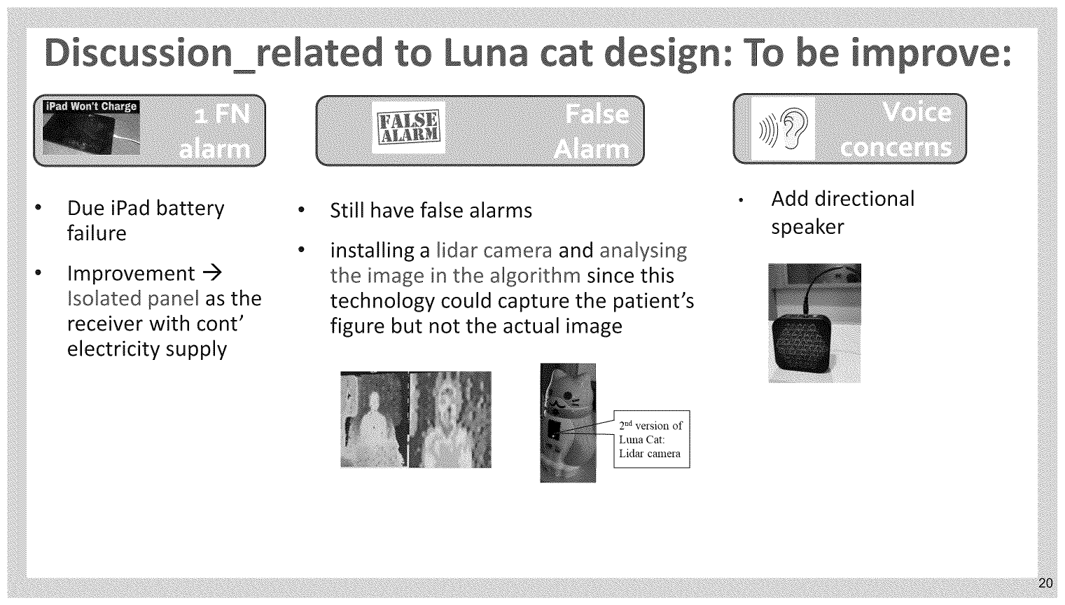

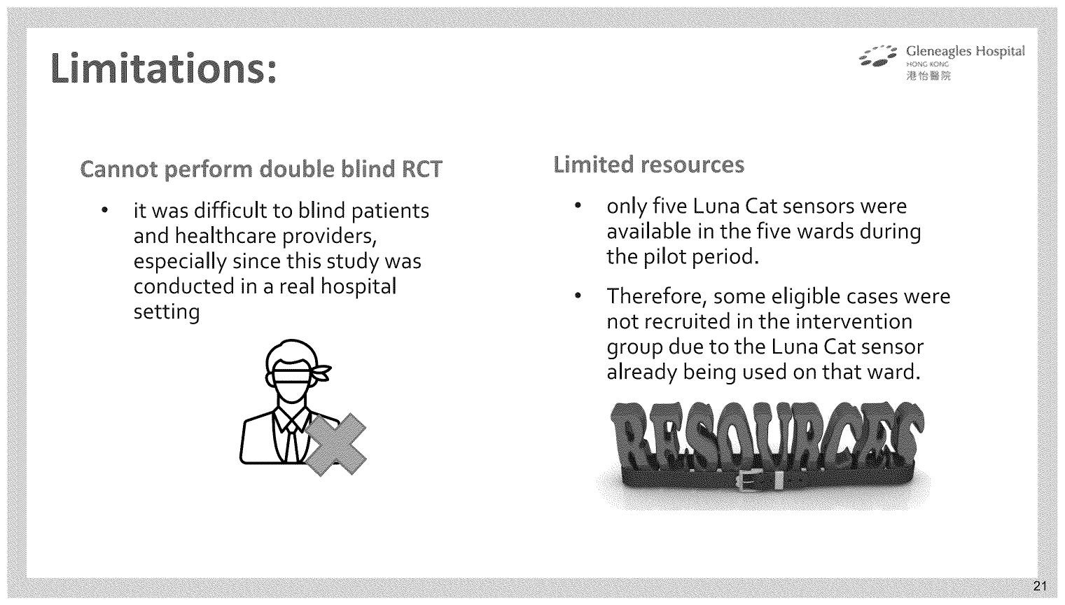

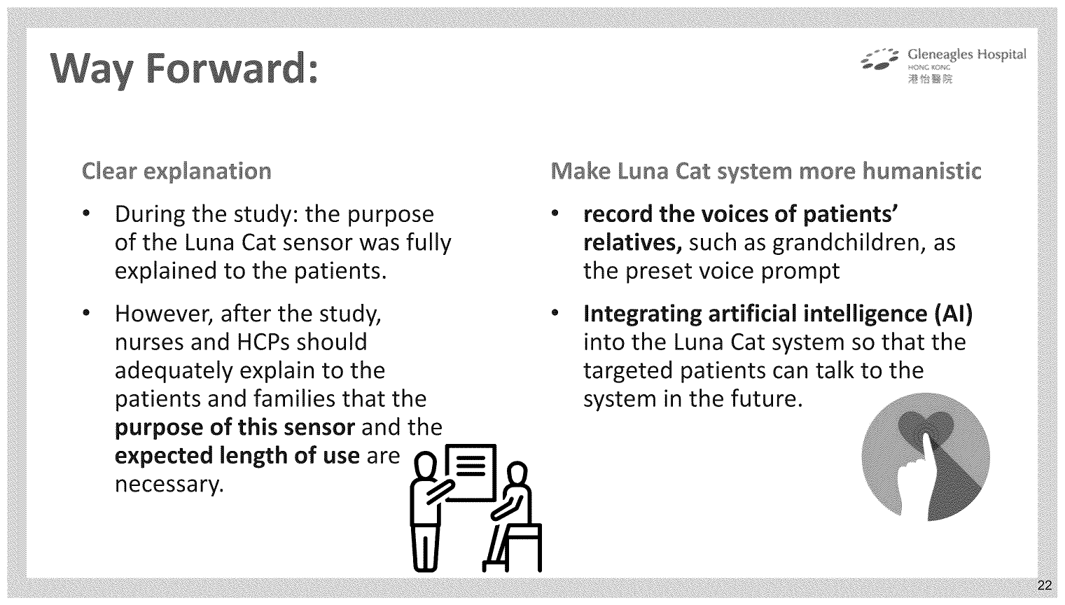

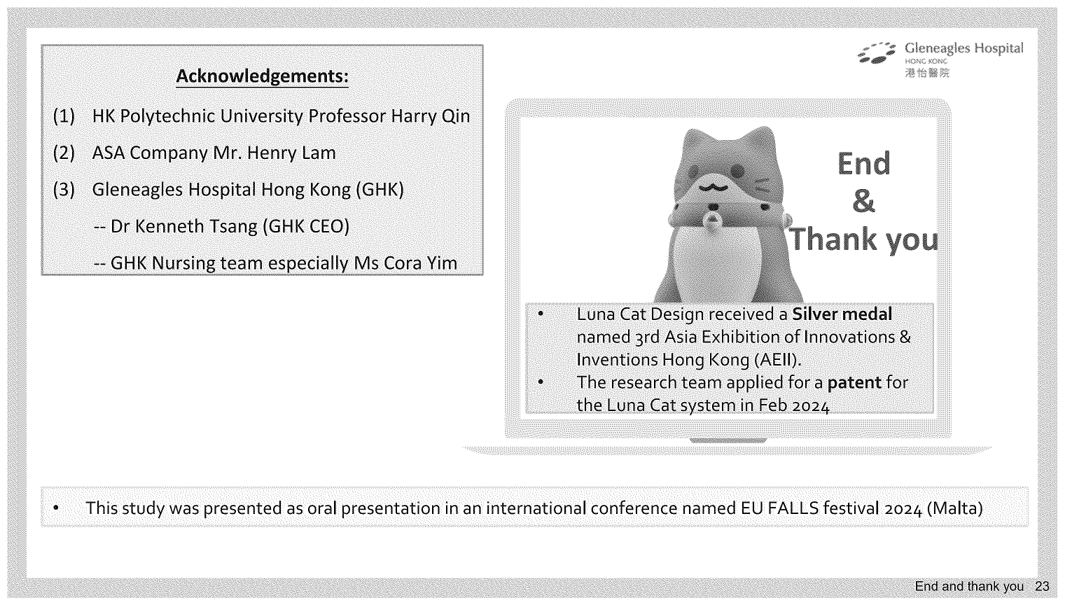

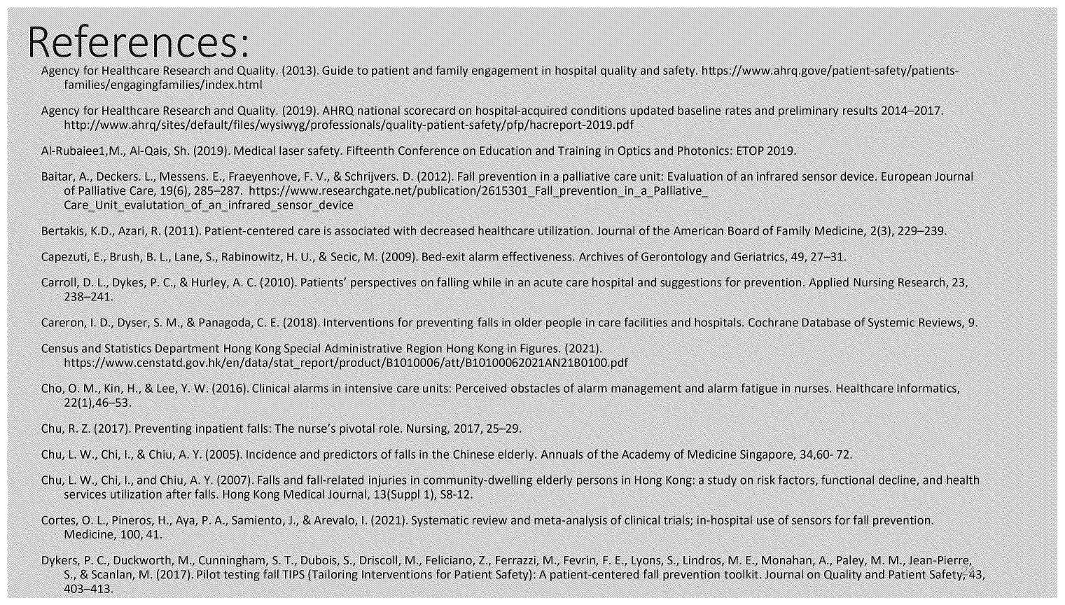

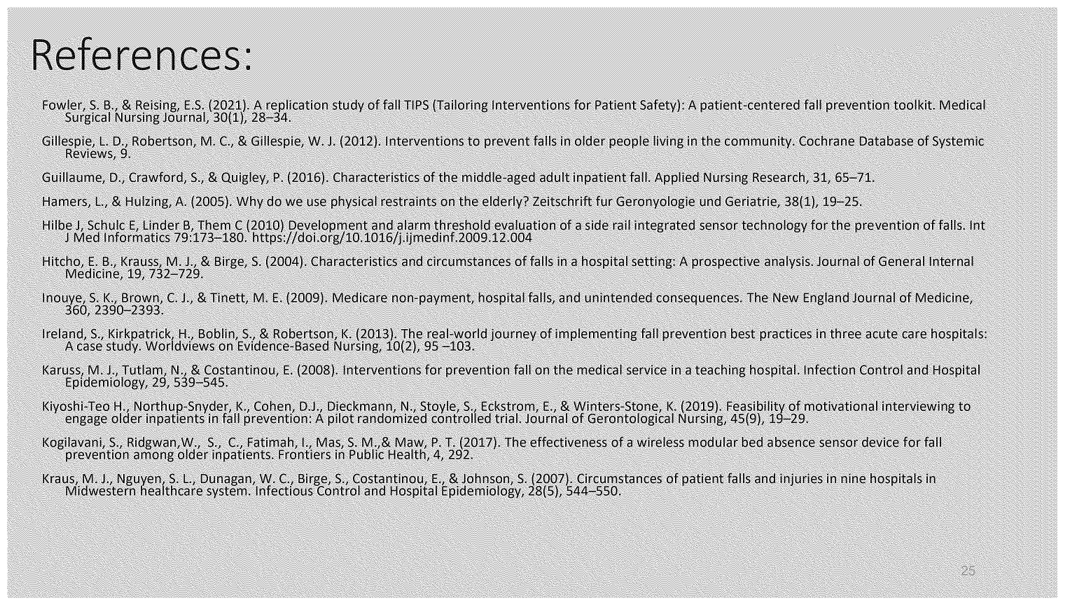

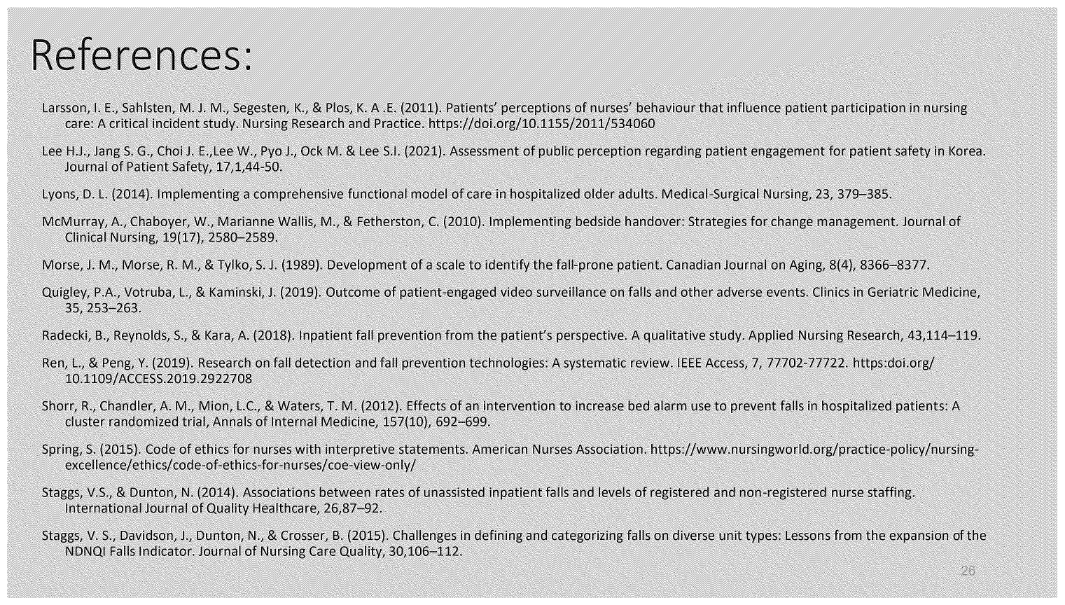

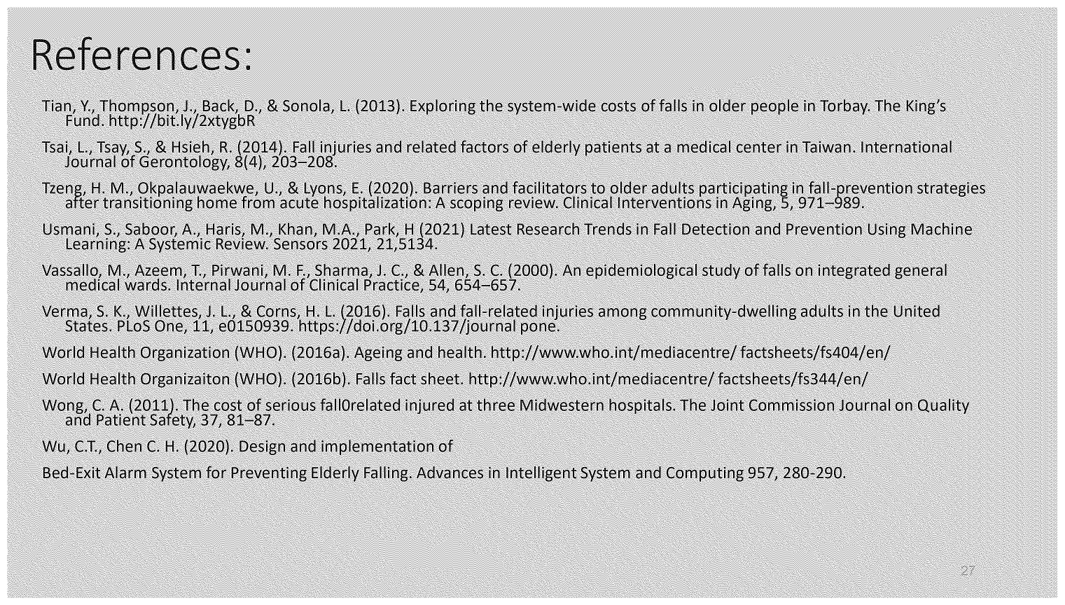

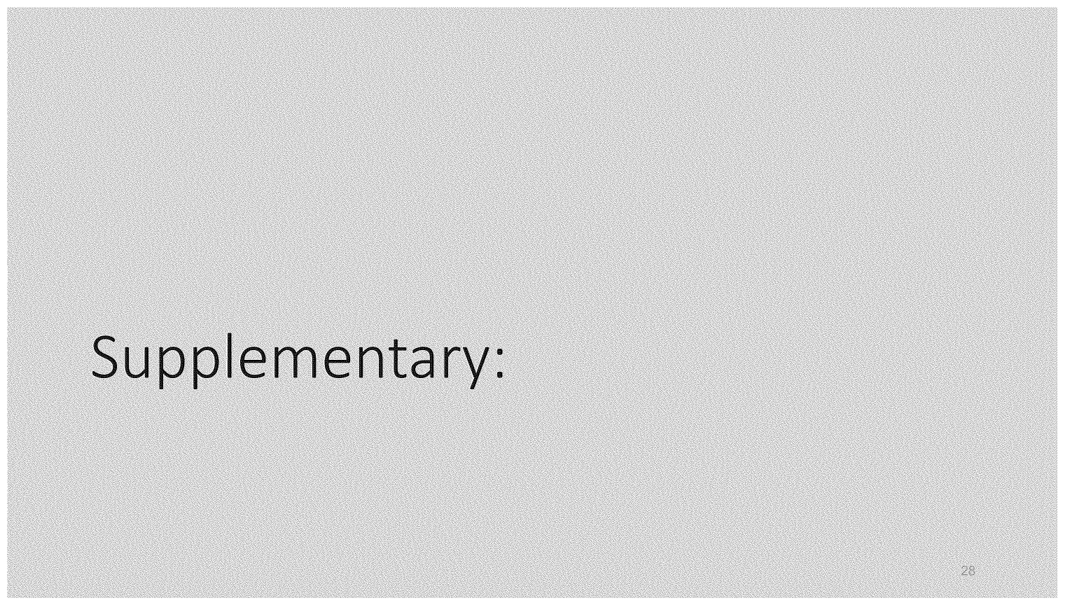

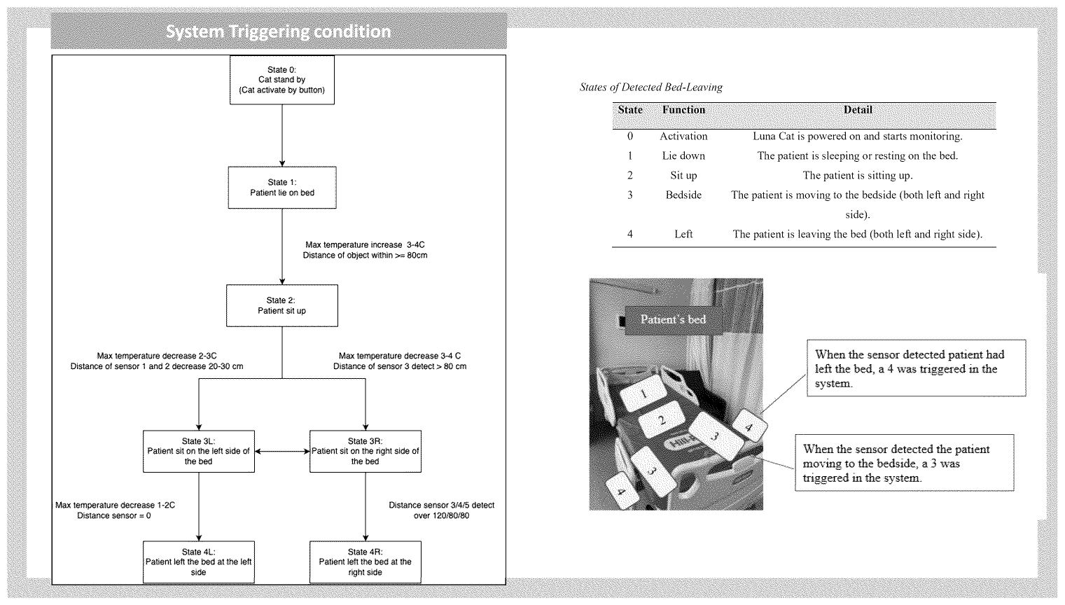

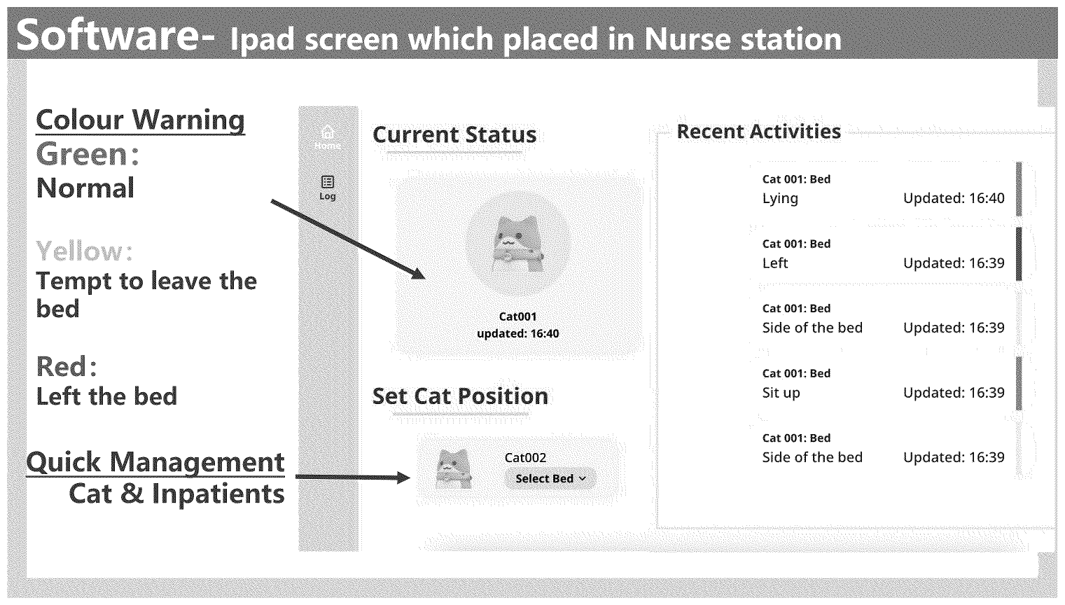

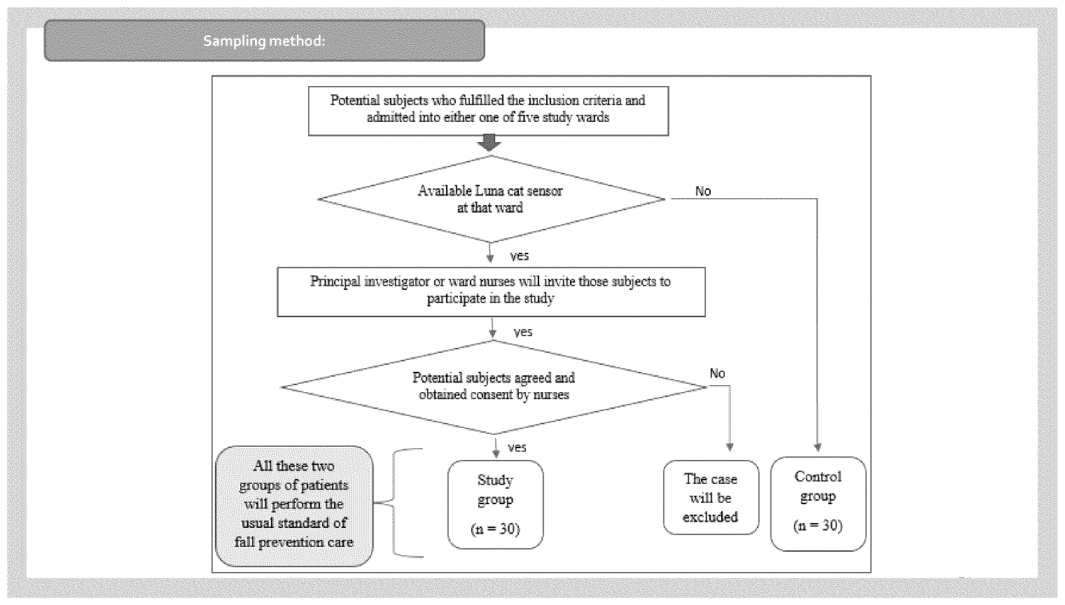

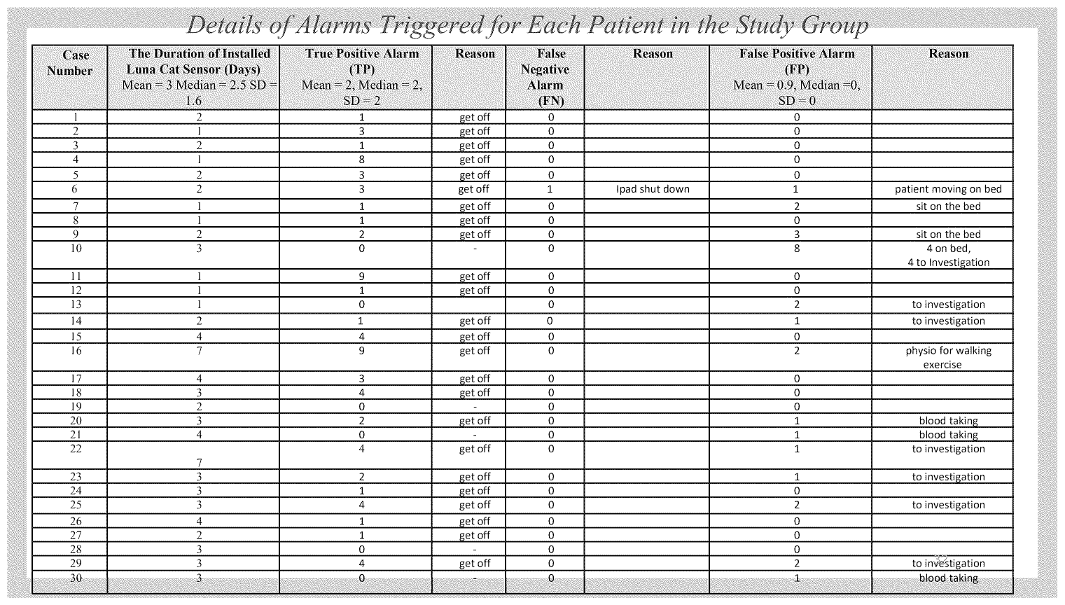

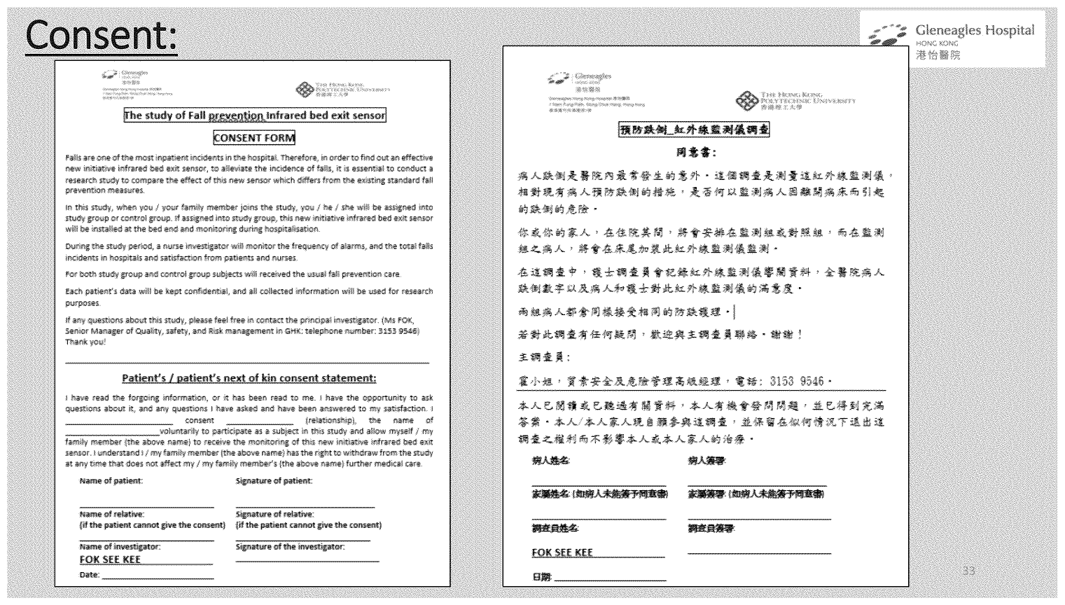

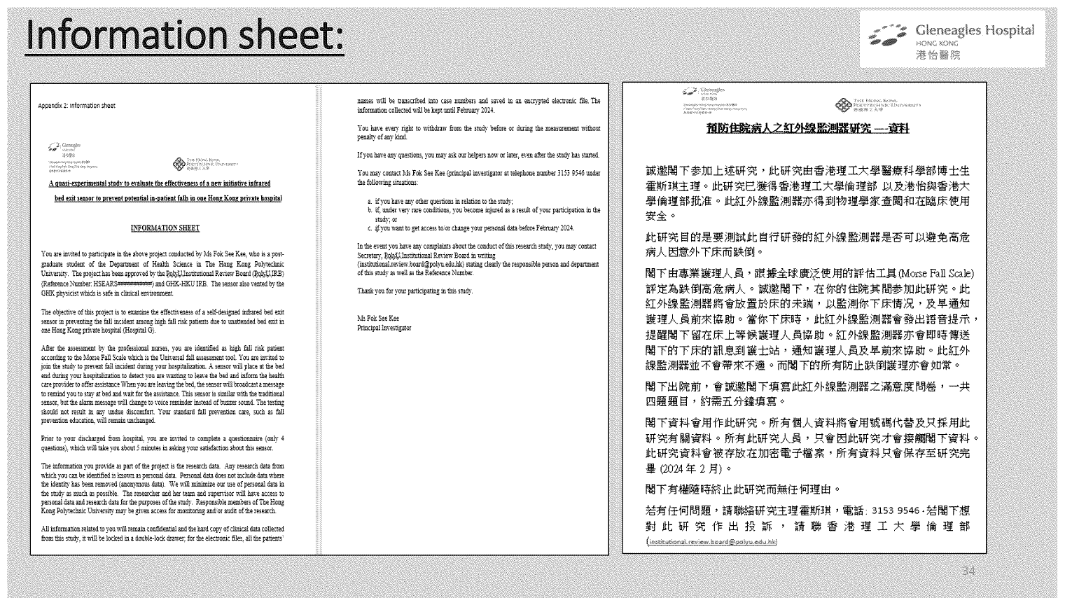

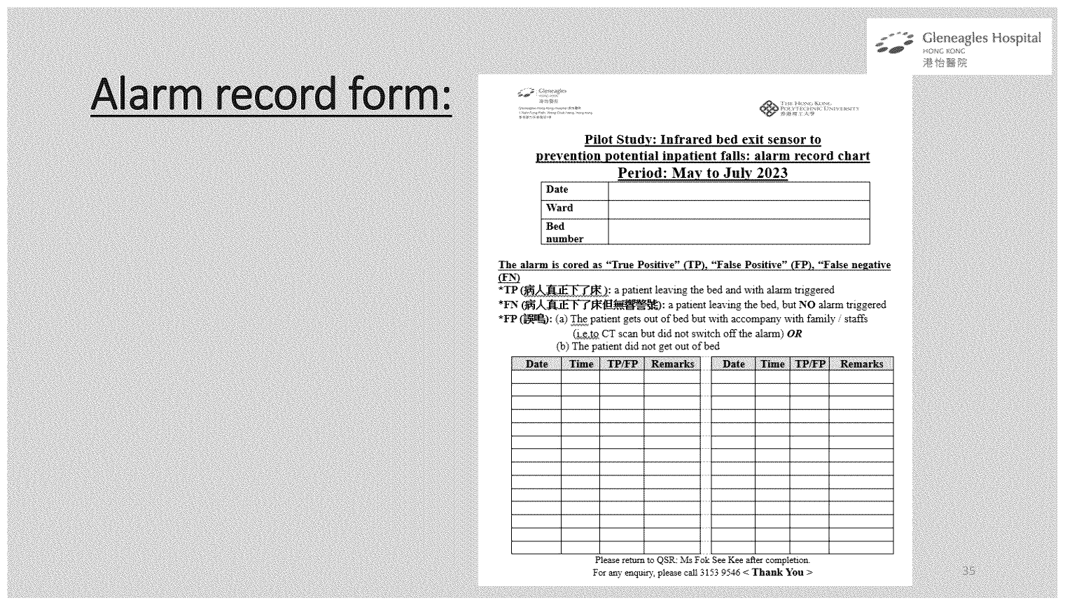

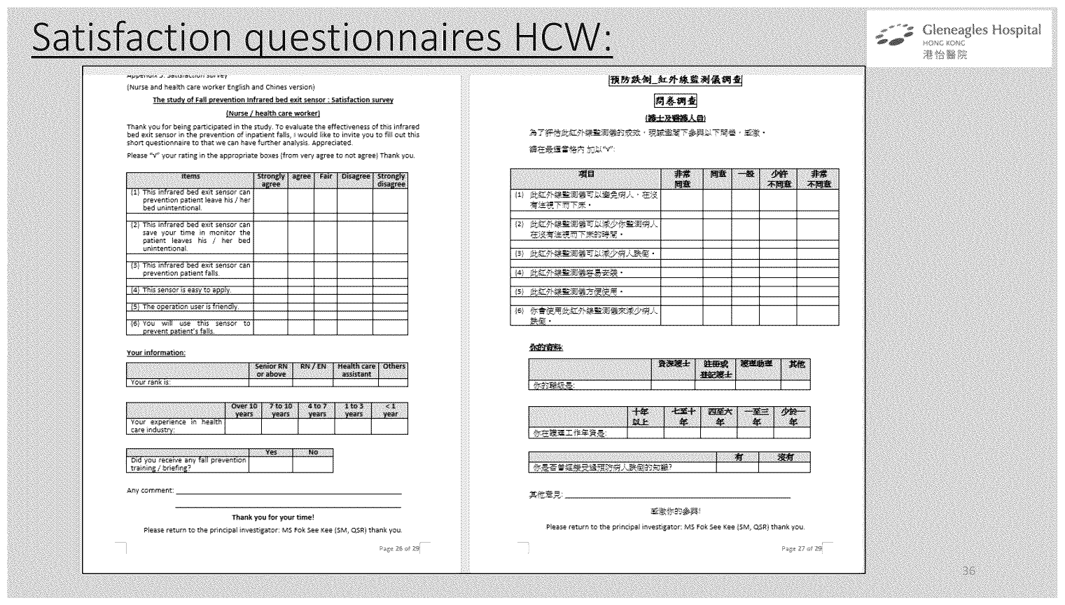

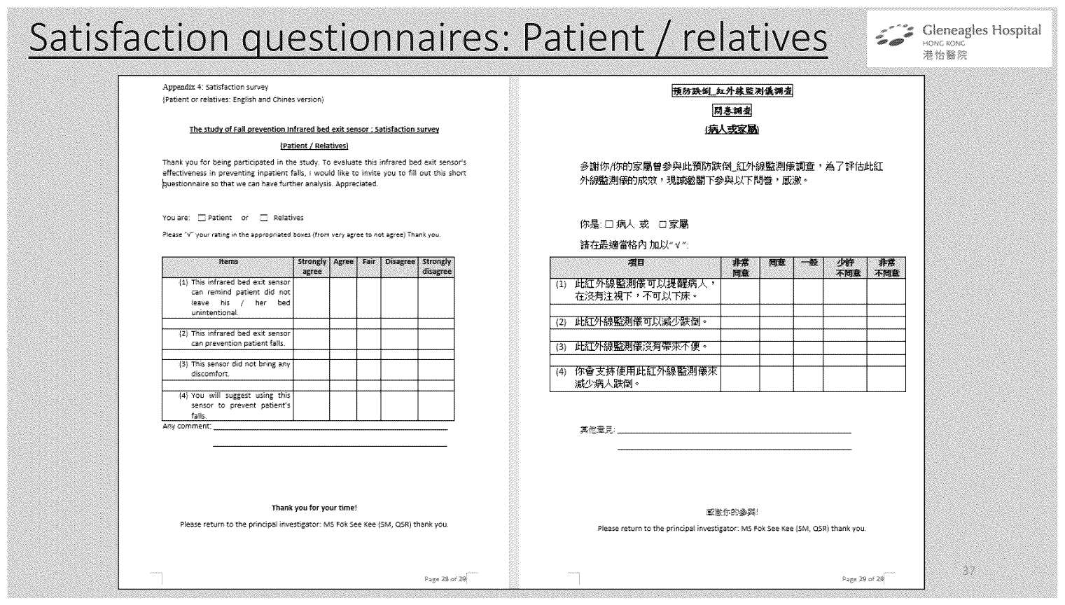

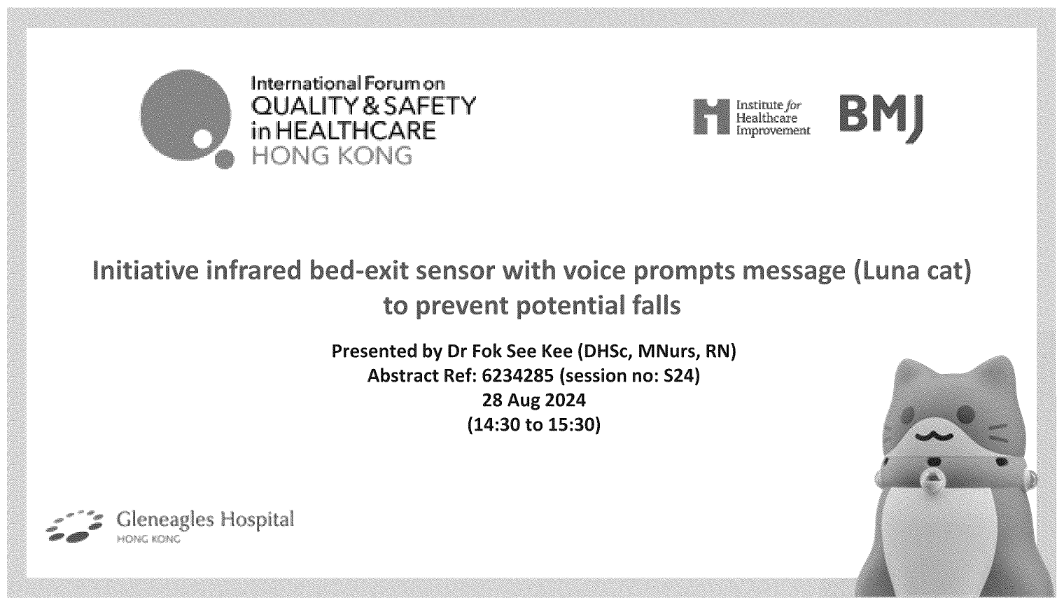

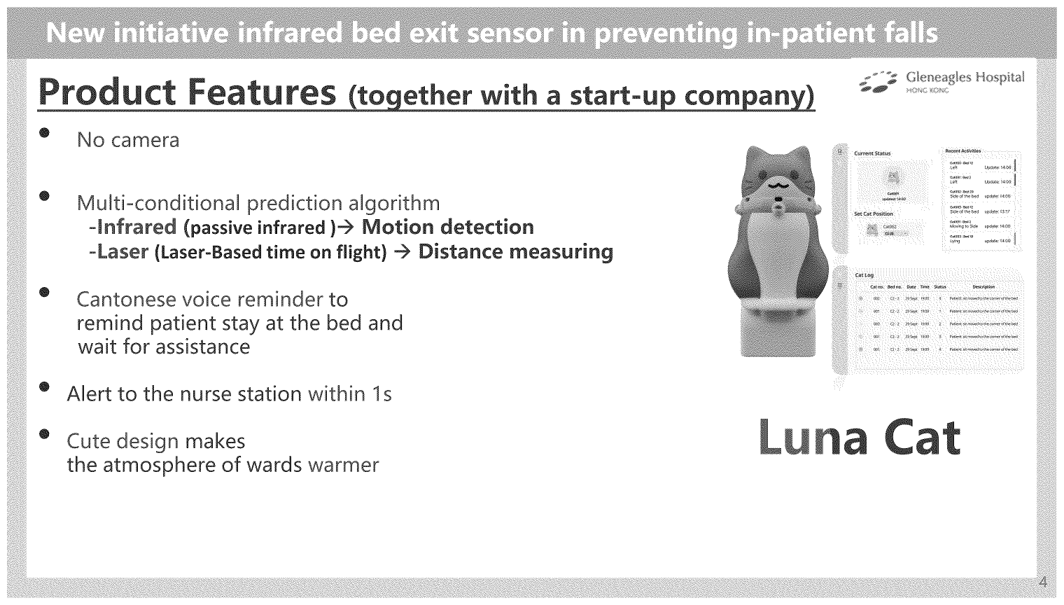

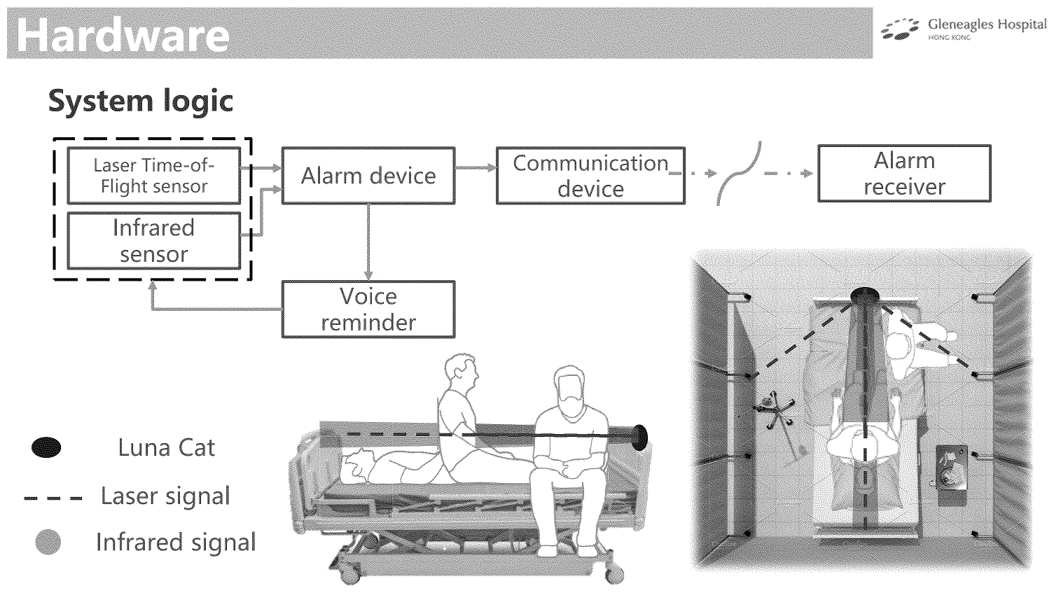

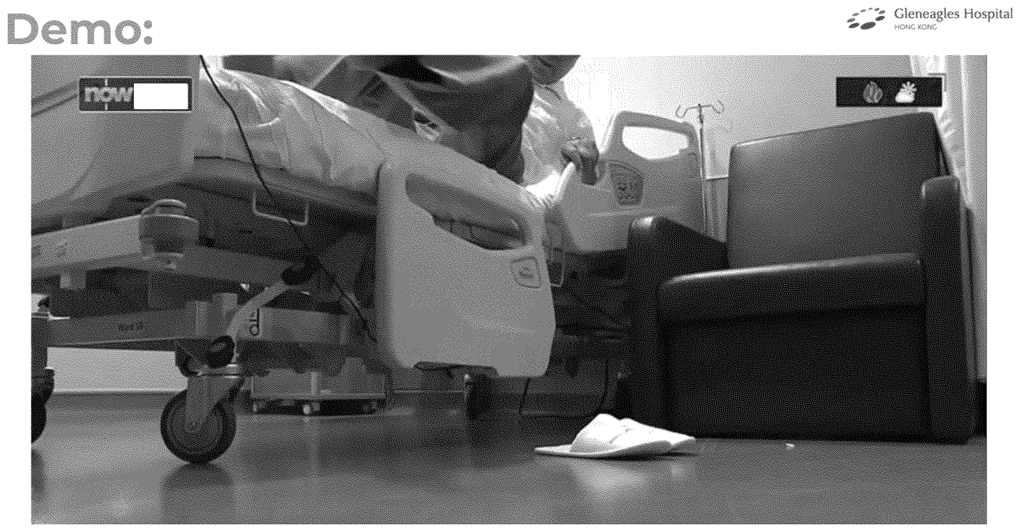

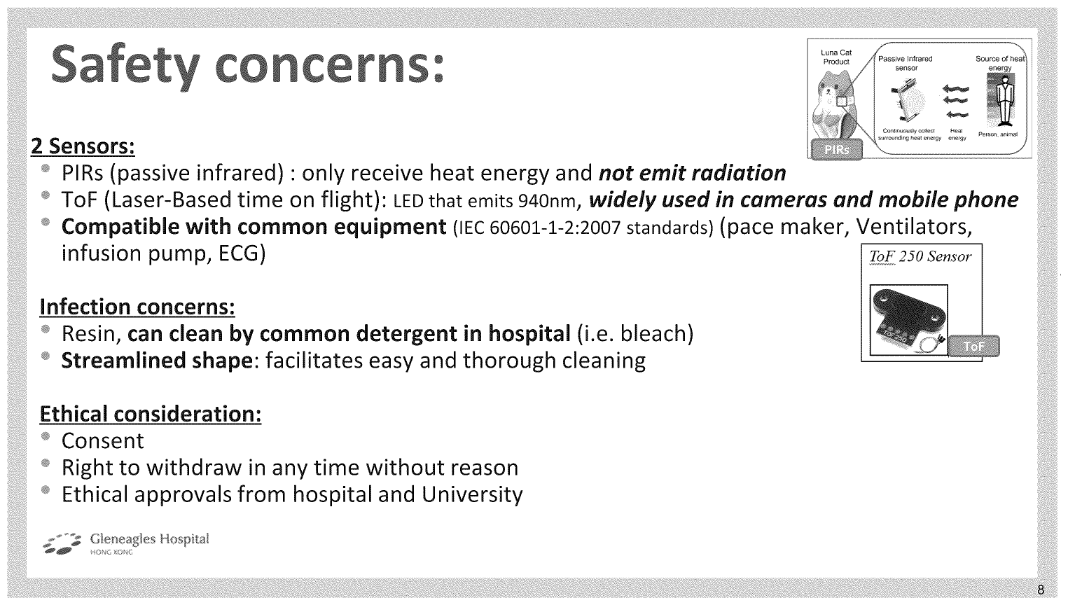

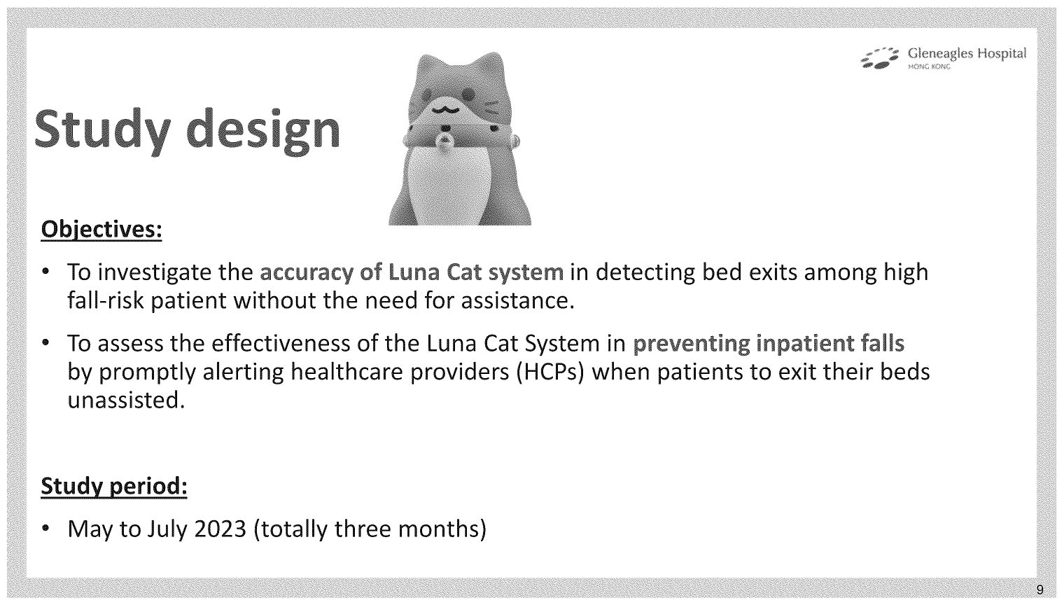

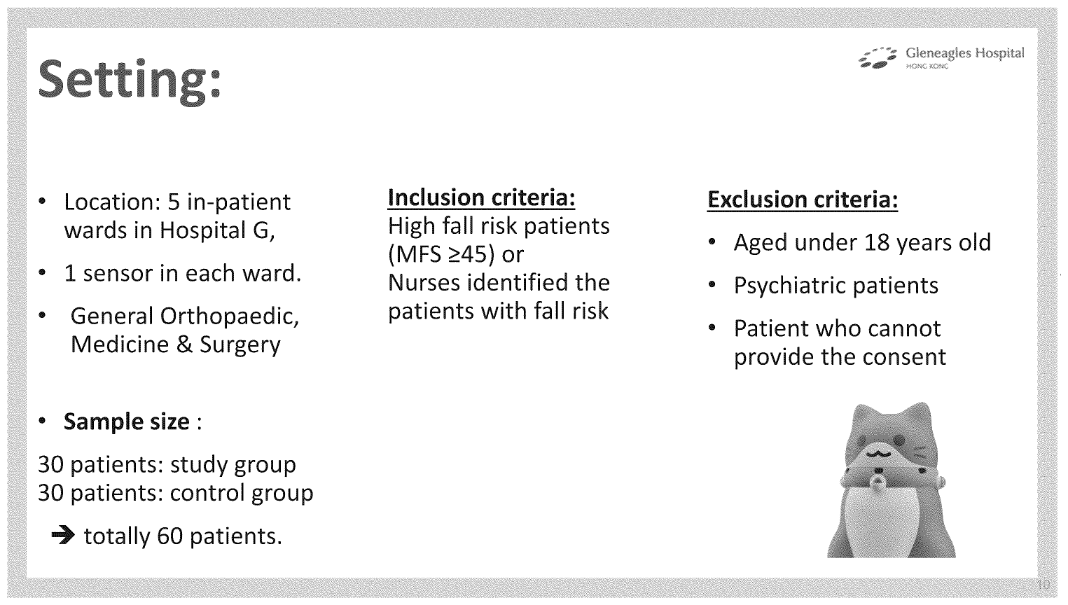

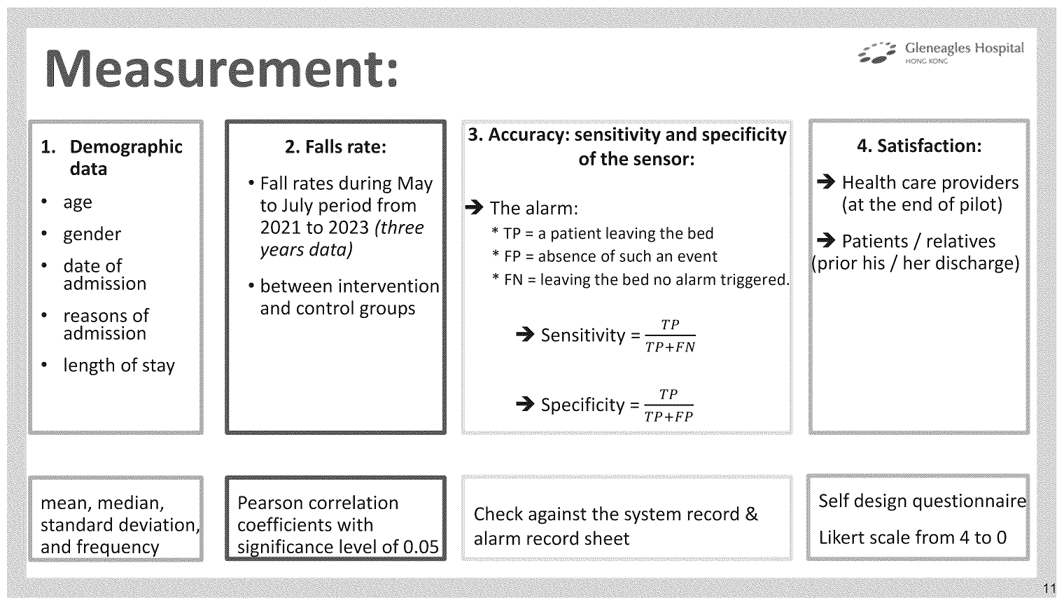

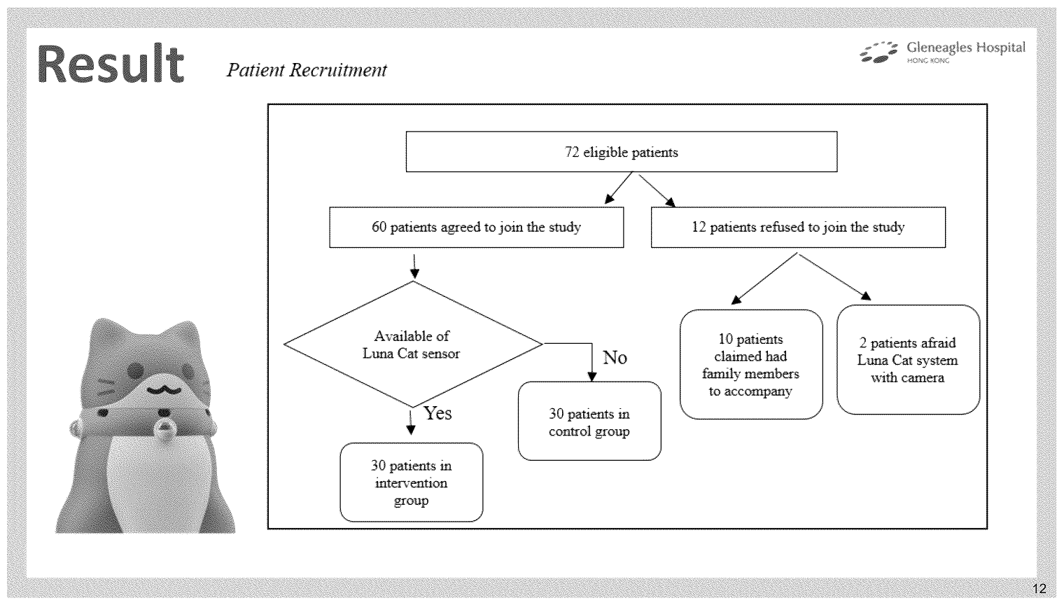

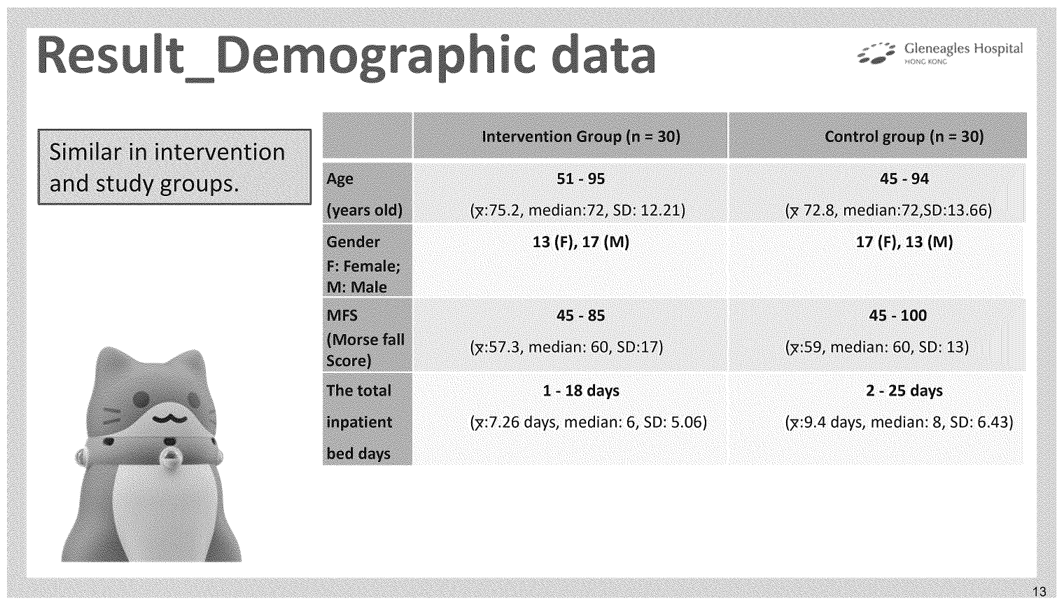

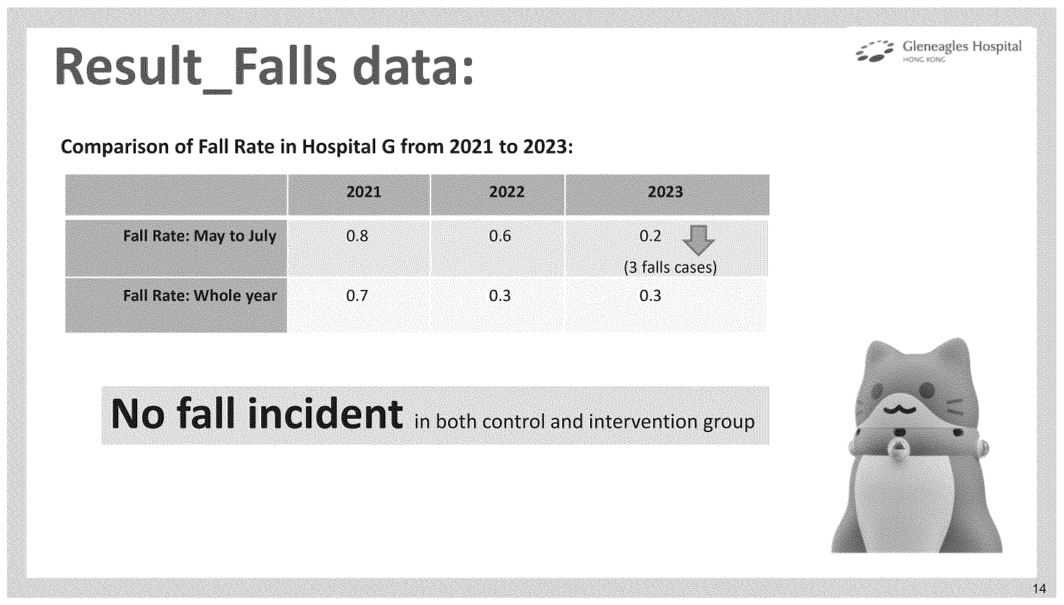

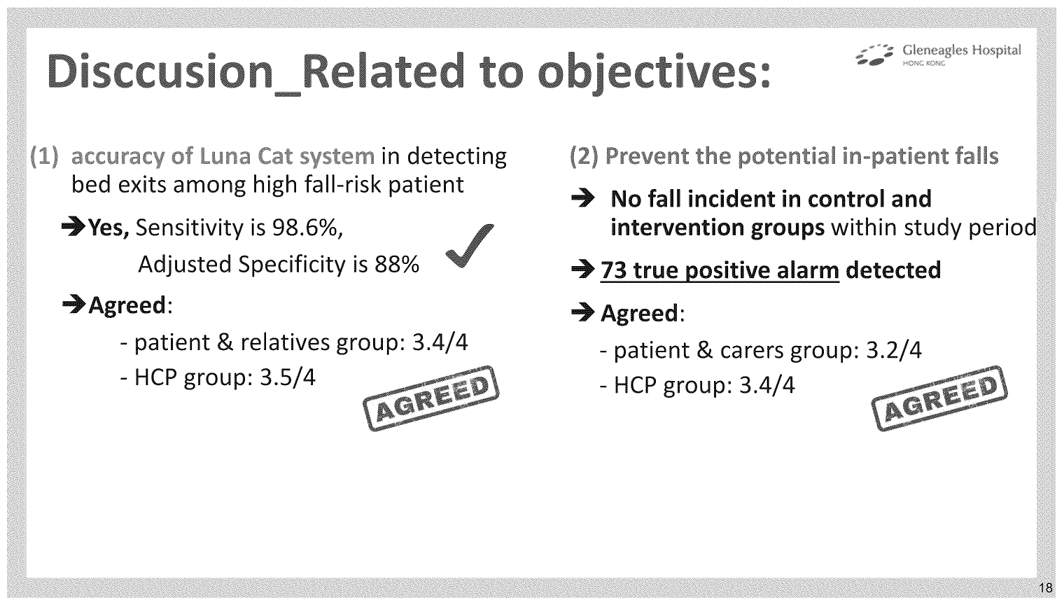

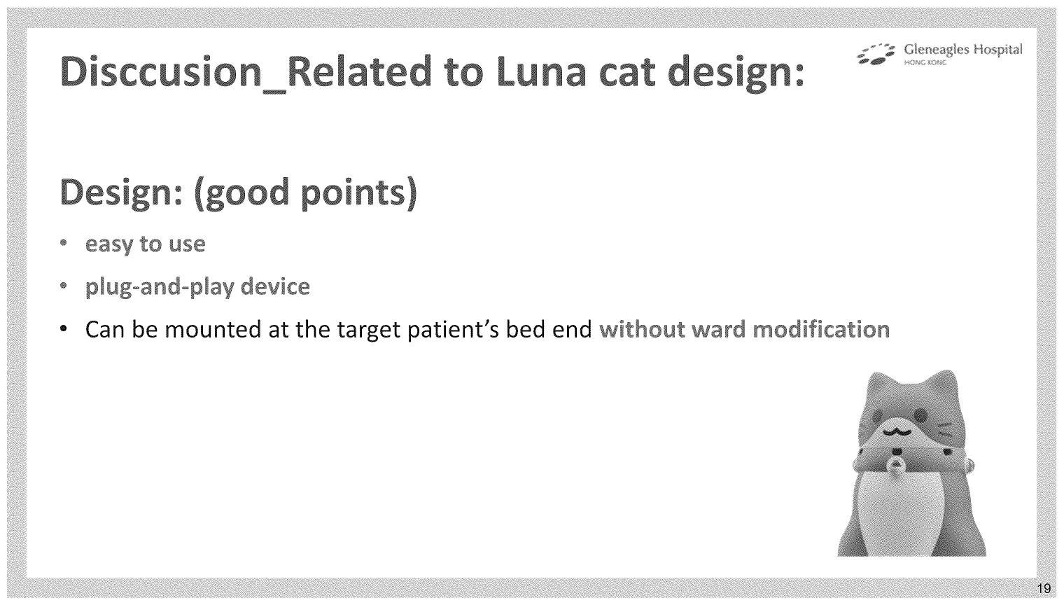

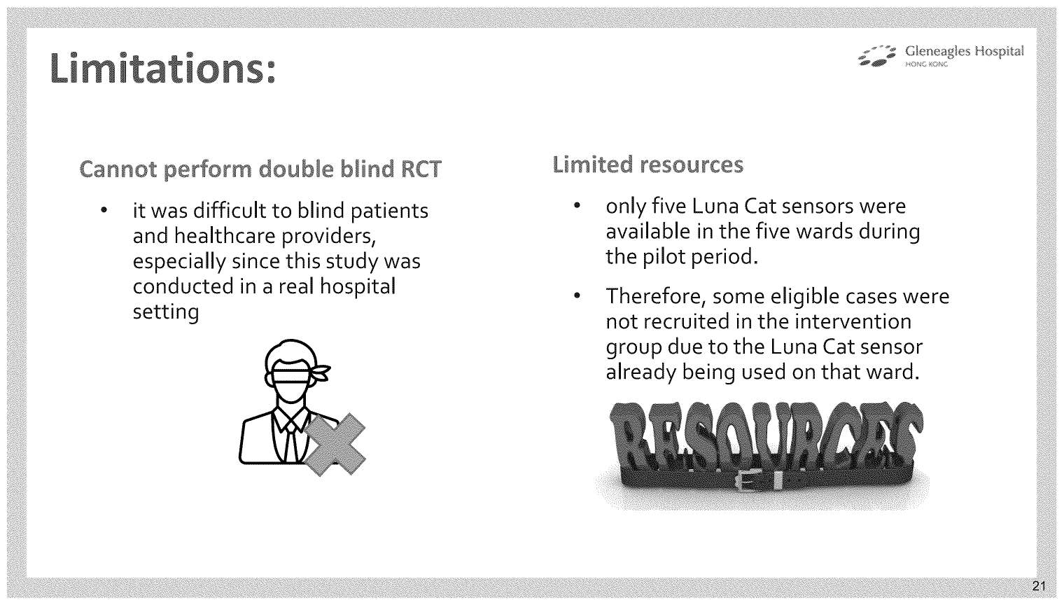

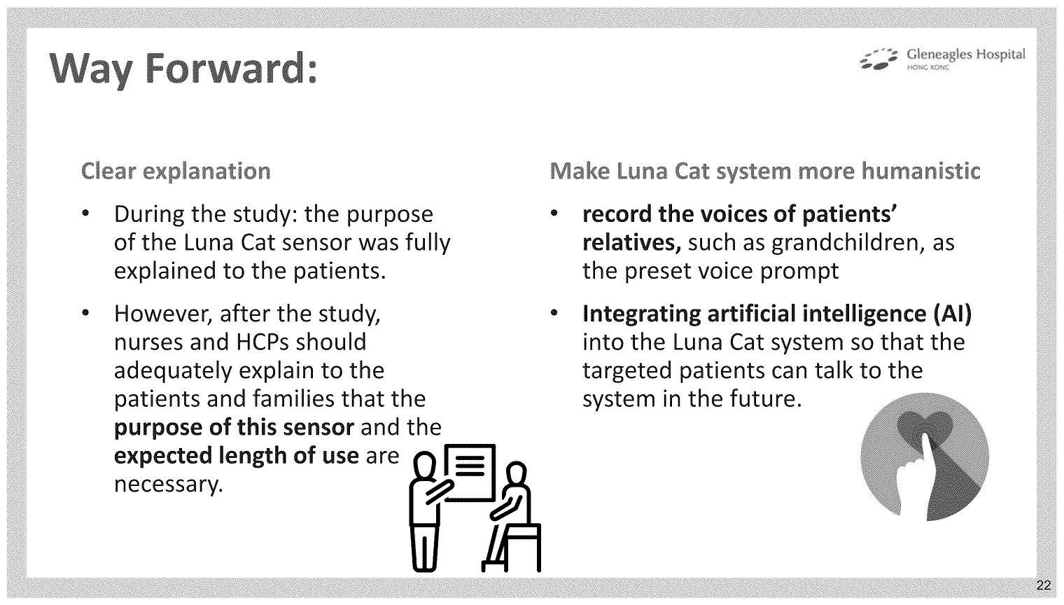

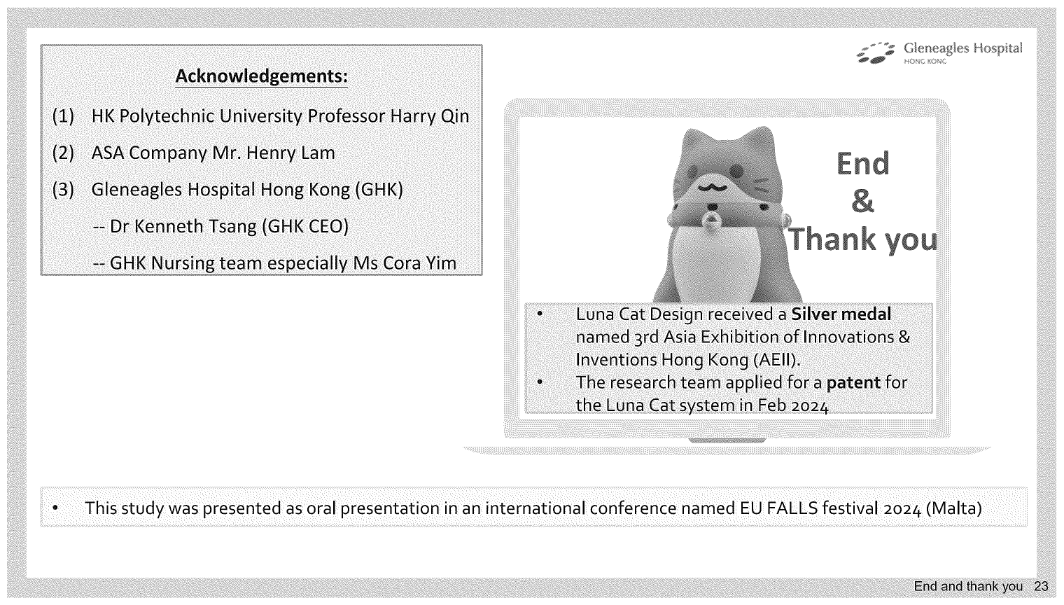

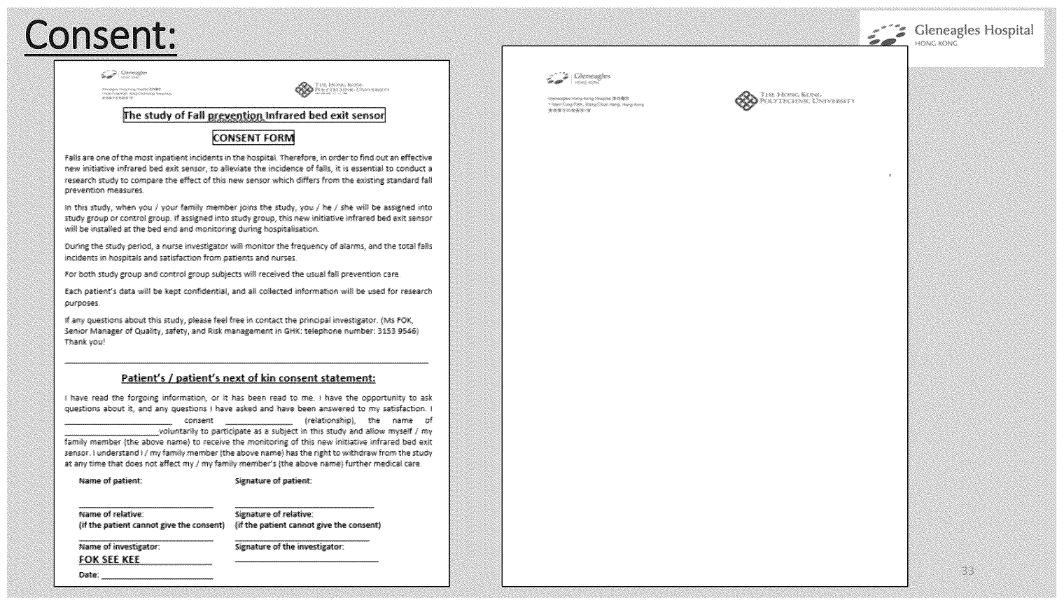

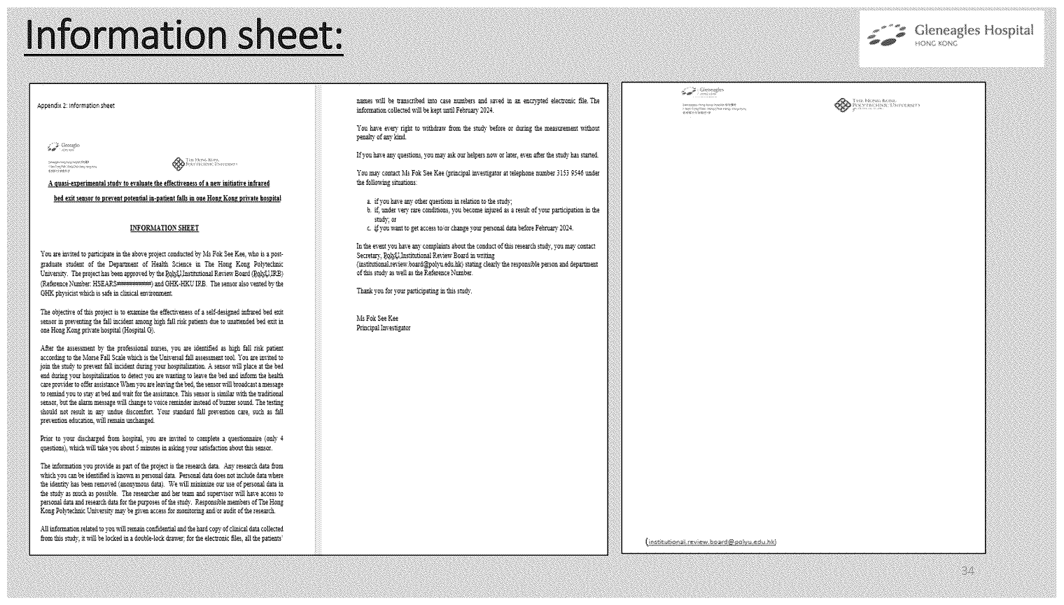

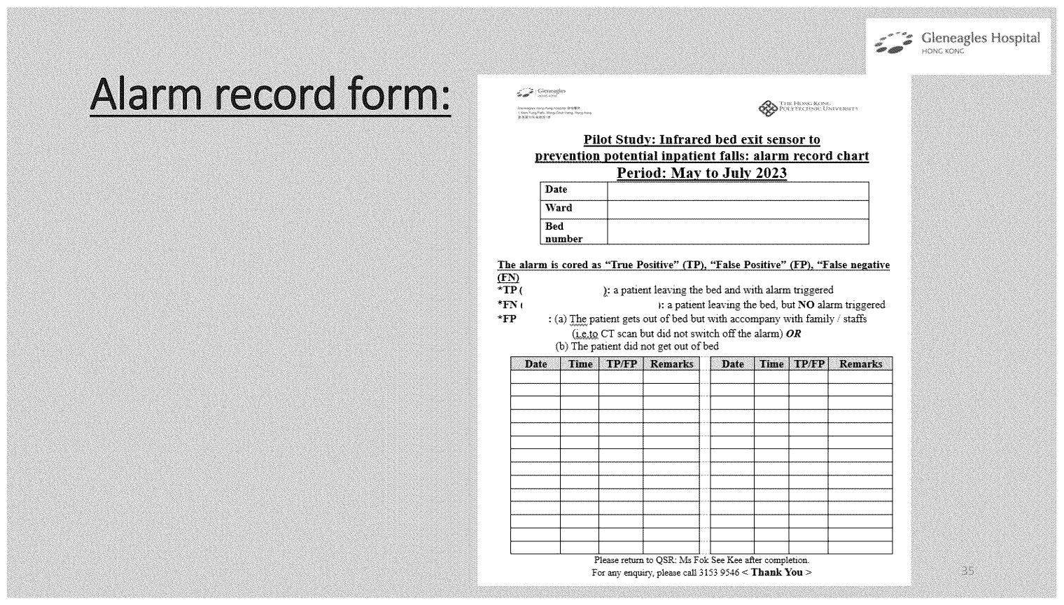

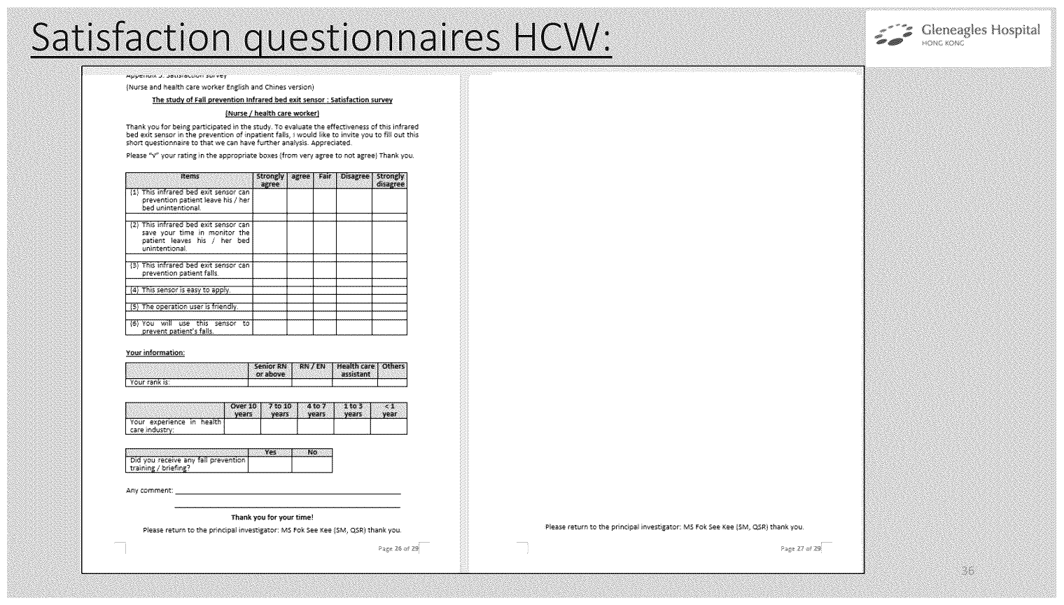

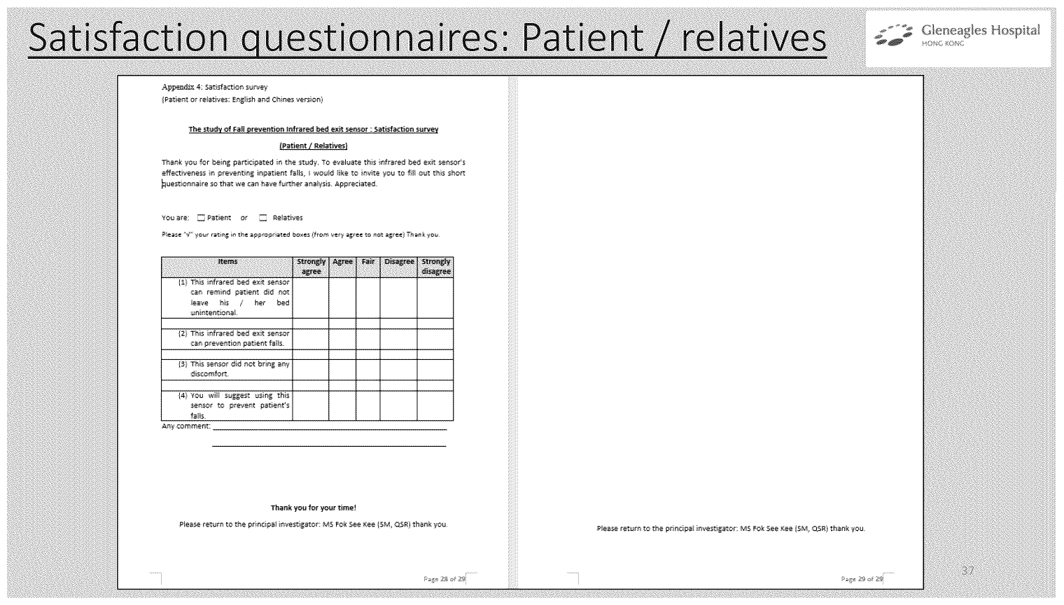

According to a first aspect of the present disclosure, there is provided an bed exit monitoring system for accurately spatially detecting a position state of a human body on the bed. Referring to FIG. 7, an bed exit monitoring system includes a distance sensor array, a thermal sensor, a processing unit, and a first alarm unit.

The measurement range of the distance sensor array covers the whole bed body and comprises distance sensors and a sensor array module, each distance sensor measures the distance between the human body and the sensor in different sections and outputs a corresponding distance signal; the sensor array module receives and processes the distance signal output by each distance sensor and outputs a distance signal matrix according to all the distance signals to create a comprehensive and detailed representation of the patient's spatial environment.

The thermal sensor is configured to measure a temperature of the bed body and output a corresponding temperature signal.

The processing unit is in communication with the distance sensor array and the thermal sensor, and is configured to process the distance signal matrix of the distance sensor array and the temperature signal of the thermal sensor to determine the position of the human body, thereby detecting the possibility of the human body leaving the bed and outputting the position state signal of the human body.

The first alarm unit is in communication connection with the processing unit and is operable to identify the position state signal output by the processing unit and make corresponding feedback.

In an embodiment, the distance sensor may be a ToF lidar sensor, a normal lidar sensor, an infrared sensor, a millimeter-wave radar, a phase difference sensor, a triangulation sensor, or another conventional distance sensor. Preferably, the distance sensor may be a ToF lidar sensor. The ToF lidar sensor can capture measurements at high speed, enabling real-time monitoring and detection of human motion, and such fast data acquisition is crucial for applications that require quick response time to ensure patient safety. In addition, ToF Lidar sensor is less affected by external environmental factors such as ambient light or weather conditions, making them more reliable in various environments, which can operate efficiently in indoor and outdoor environments, providing consistent and robust performance. Moreover, ToF Lidar sensor can provide additional information beyond distance measurements, such as object shape and orientation, which can be used to enhance detection and recognition of specific objects or body movements, supporting more advanced monitoring and intervention functions. Furthermore, ToF lidar technology has become more widely available and affordable in recent years, with a range of commercial options on the market, which accessibility makes it easier to integrate ToF lidar into the bed exit system, thereby potentially reducing development costs and improving overall affordability.

In an embodiment, the distance sensors have different detection angle, and the detection angle of the distance sensor array covers the entire bed body and the surrounding area of the bed body, so as to monitor the position and movement of the human body and determine the presence of caregivers around the bed. Optionally, 2-12 distance sensors are provided. However, those skilled in the art should also understand that the number of the distance sensors may be set as required, and is not limited by the number of installation provided in this embodiment.

Optionally, two rows of distance sensors are provided, and each row includes 3 distance sensors. Referring to FIG. 6, the measurement angle of the distance sensor are controlled, so that the measurement range of the distance sensor array covers the whole bed body and the surrounding area of the bed body.

In an embodiment, a bed exit monitoring system further includes a temperature sensor, where the temperature sensor is communicatively connected to the processing unit, and is configured to measure a room temperature/an ambient temperature and output a corresponding temperature signal. The processing unit compares the temperature sensor reading with the thermal sensor reading to determine if the bed is occupied, that is, when the thermal sensor reading is close to the temperature sensor reading (within 2 degrees Celsius), it indicates that the bed is not occupied; when the thermal sensor reading differs greatly from the temperature sensor reading (more than 2 degrees Celsius), it indicates that the bed is occupied. In general, when the bed is occupied, the thermal sensor reading approaches the human body temperature (36±2 degrees Celsius). The temperature sensor is adopted to measure the room temperature/ambient temperature as the contrast temperature of the thermal sensor, the processing unit realizes automatic self-calibration by comparing the room temperature and the bed range temperature to ensure the monitoring accuracy.

In an embodiment, within the processing unit, a classification module is programmed to classify a human body position into safe, abnormal, and high-risk states based on the distance signal matrix of the distance sensor array and the temperature signal output by the thermal sensor and the temperature sensor.

Specifically, the classification module is a software coding run in the processing unit, it classifies the human position state based on the spatial distance readings from the sensor array and also the temperature difference between ambient environment and human body, and then transforms a 3D mapping (x, y, and depth) for motion tracking and object position classification.

In an embodiment, the distance sensor array may measure a human body distance around the bed body, and the processing unit determines, according to the human body distance around the bed body measured by the distance sensor array, whether a caregiver exists around the bed body.

When the patient position is in an abnormal state or a high-risk state, the first alarm unit sends out corresponding auditory information to remind and intervene the patient, the first alarm unit can deliver a customized message based on the detected abnormal human body action, and the personalized method is beneficial to effectively communicate with the human body and remind the human body to avoid potential risky motion, and promotes the compliance and safety of the human body.

In an embodiment, a bed exit monitoring system further includes a remote device, a second alarm unit is disposed in the remote device station, and the second alarm unit is communication with the processing unit, and is configured to identify a position state signal output by the processing unit, and feedback corresponding prompt information in different position states, so that a guardian or a caregiver can respond in time, thereby reducing the possibility of human accidents or walking.

Optionally, the remote device includes a console and/or a user device in at least one embodiment. The console may be disposed at a nurse station, and the user equipment may be a mobile device such as a smart phone or a tablet computer. The amount of consoles and user devices can be set as required.

Optionally, the remote device further includes a display unit, and the display unit is in communication connection with the processing unit, and is configured to present a real-time human body position state, so that a guardian or a caregiver can know patient dynamics conveniently.

In an embodiment, the second alarm unit may generate an audible alarm and/or a visual alarm. Optionally, the visual alarm may use different colors to represent different human body position states. For example, a green prompt is sent in a safe state; a yellow prompt is sent in an abnormal state; and a red prompt is sent in a high-risk state. The second alarm unit does not release the audible alarm in the safe state, and emits the audible alarm in the abnormal state and high risk state. In the system standby state, the remote device does not make a sound prompt and displays gray.

In an embodiment, a bed exit monitoring system further includes a communication module, and the communication module is configured to transmit a position state signal of a human body. The communication module may transmit data using a wired or wireless link. Optionally, the communication module includes, but is not limited to, WiFi, Bluetooth, and the like.

In an embodiment, a bed exit monitoring system further includes a cloud server, the processing unit uploads the position state signal to the cloud server, and the cloud server sends the position state signal to a remote device.

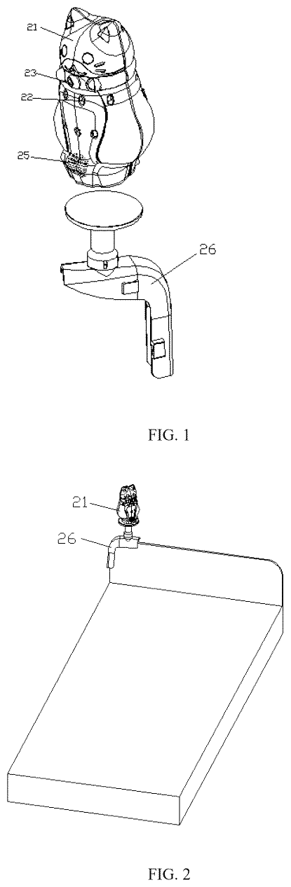

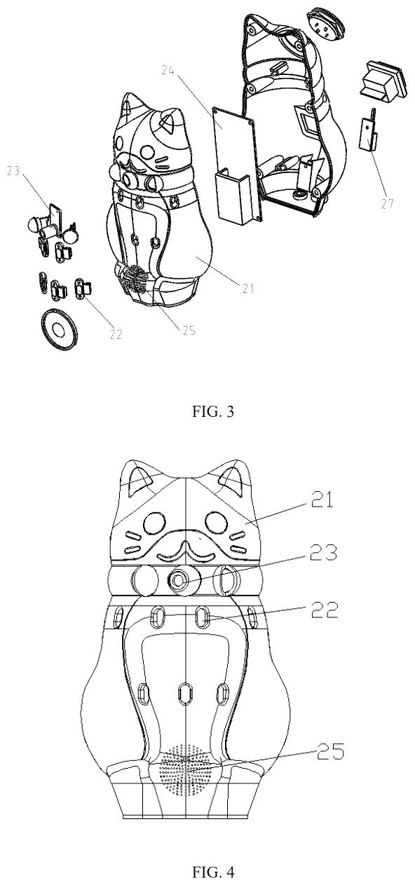

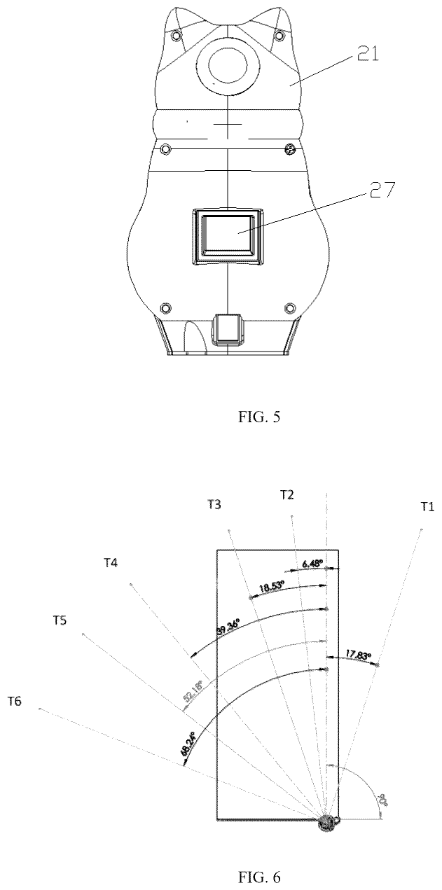

According to a second aspect of the present disclosure, referring to FIG. 1-FIG. 5, a bed exit monitoring device is provided, which is disposed on a frame of a bed and includes a case 21, a distance sensor array 22, a thermal sensor 23, a print circuit board assembly (PCBA) 24, a speaker assembly 25 and a bracket 26. A chamber is disposed inside the case 21, and a switch is disposed outside the case 21. The distance sensor array 22 and the thermal sensor 23 are arranged on the case 21 and face the bed body. The measurement range of the distance sensor array 22 covers the whole bed body and comprises distance sensors and a sensor array module, and each distance sensor measures the distance between the human body with the sensor in different sections and outputs a corresponding distance signal; the sensor array module receives and processes the distance signal output by each distance sensor and outputs a distance signal matrix according to all the distance signals to create a comprehensive and detailed representation of the human body spatial environment. The thermal sensor 23 is operable to measure the temperature in the range of the bed body and output a corresponding temperature signal. The print circuit board assembly 24 is arranged in the chamber of the case 21 and comprises a processor, the processor is in communication connection with the distance sensor array 22 and the thermal sensor 23, and is configured to process the distance signal matrix of the distance sensor array 22 and the temperature signal of the thermal sensor 23 to determine the position of the human body so as to detect the possibility that the human body leaves the bed and output a position state signal. The speaker assembly 25 is arranged on the case 21 and is in communication connection with the processor, and a corresponding sound prompt is fed back according to the position state signal sent by the processor. The bracket 26 is arranged on the bed frame.

The case 21 may be provided as a spherical, cuboid or other three-dimensional structure. Optionally, the case 21 is provided in a shape of a cat to improve the aesthetics of the device. The case 21 includes a front case and a rear case, and the front case and the rear case are detachably connected to facilitate inspection and maintenance of the device.

The device switch is arranged on the rear case, and controls the opening and closing of the bed exit monitoring device.

The distance sensor array 22 and the thermal sensor 23 are disposed on the front case. Optionally, the distance sensor array 22 includes 2-12 distance sensors. There is 1 thermal sensors 23. Preferably, two rows of distance sensors are provided, and each row includes 3 distance sensors.

A classification module is set in the processor. The processor determines a human position based on the distance signal matrix from the sensor array 22 and the temperature signal output by the thermal sensor 23, and classifies a human body position into safe, abnormal, or high-risk states.

The speaker assembly 25 includes a speaker and a volume button, the speaker is disposed on the front case, the volume button is disposed on the rear case 21, and the volume of the speaker is controlled by the volume button.

In an embodiment, a bed exit monitoring device is provided with a power supply, and the power supply may be an external power supply or a battery assembly.

In an embodiment, a bed exit monitoring device further includes a temperature sensor 27, where the temperature sensor 27 is communicatively connected to the processor, and is configured to measure a room temperature/an ambient temperature. The temperature sensor 27 is disposed on the rear case. The processor compares the temperature sensor reading with the thermal sensor reading to determine if the bed is occupied.

In an embodiment, a bed exit monitoring device further includes a remote device, a second alarm unit is disposed in the remote device, and the second alarm unit is in communication connection with the processor, and is configured to identify a position state signal output by the processor, and feed back corresponding prompt information in different human body position states. The second alarm unit can generate an auditory alarm and/or a visual alarm, so that a guardian or a caregiver can react in time.

Optionally, the remote device may include a console and/or a user device. The console may be disposed at a nurse station, and the user equipment may be a mobile device such as a smart phone or a tablet computer. The amount of console and user device can be set as required.

Optionally, the remote device further includes a display unit, and the display unit is in communication connection with the processor, and is configured to present real-time human body movement data and a human body position state, so that a guardian or a caregiver can know human body dynamics conveniently.

In an embodiment, an bed exit monitoring device further includes a cloud server, the cloud server is in communication connection with the processor and the remote device respectively, the processor uploads the position state signal to the cloud server, and then the cloud server sends the position state signal to the remote device.

In an embodiment, the bracket 26 includes a clamping part and a top plate, the clamping part is designed to be fitted to a bed frame, the top plate is disposed on the clamping portion, and the case 21 may be connected to the top plate by magnetic attraction, clamping, or other detachable connection manners.

It should be understood that a bed exit monitoring device may not only be mounted on a bed, but also may be mounted on other supports such as a wheelchair, a roller bed, a sofa, a chair or a reclining chair, and a human body lies on the supports.

According to a third aspect of the present invention, there is provided an bed exit monitoring method for the system according to the first aspect of the present invention and/or the device according to the second aspect of the present invention, comprising the following steps:
S1, installing the bed exit monitoring device on the bed frame;
S2, witching on the device and connecting to a cloud server;
S3, measuring, by the distance sensor array, the distance between human body and the distance sensor array; and measuring, by the thermal sensor, the temperature within the bed body; and measuring, by the temperature sensor, the room temperature/ambient temperature;
S4, determining, by the processor, a human body position based on the spatial distance readings from the sensor array and also the temperature difference between the thermal sensor reading and the temperature sensor reading, and determining which human body position state the human in;
S5, uploading, by the processor, the human body position states to a cloud server;
S6, sending, by the cloud server, the human body position states to the remote device;
S7, displaying, by the remote device, a human body position state, and feeding back corresponding prompt information in different states.

In an embodiment, a specific method of step S3 of the bed exit monitoring method is as follows: each distance sensor in the distance sensor array measures a distance between a human body and a sensor in different angles, and outputs a corresponding distance signal; a sensor array module of the distance sensor array receives and processes the distance signal output by each distance sensor, and outputs a distance signal matrix according to all the distance signals; and the thermal sensor is configured to measure a temperature in a range of the bed body, and output a corresponding temperature signal; and the temperature sensor is configured to measure the room temperature/ambient temperature, and output a corresponding temperature signal.

Additionally, in order to avoid false alarms caused by the presence of nursing staff, in step S3, the detection angle of the distance sensor array covers the entire bed body and the surrounding area of the bed body. The step S4 further includes: measuring, by the distance sensor array, a distance of a person around the bed body, and determining, by the processor, whether a caregiver exists around the bed body, and when the caregiver exists, not triggering an alarm.

Furthermore, in step S4, the method for determining the human body position states includes: when the results of the distance sensor array readings and the temperature difference between the thermal sensor reading and the temperature sensor reading meet a first preset condition, determining, by the processor, the position of the human body as a safe state; when the above results meet a second preset condition, determining, by the processor, the human body position as an abnormal state; when the above results meet a third preset condition, determining, by the processor, the human body position as a high-risk state; when the above results meet the fourth preset condition, determining, by the processor, that a caregiver exists around the bed body.

Optionally, the first preset condition to the fourth preset condition are set according to a spatial size of the monitoring range. The monitored bed size is ranging from 90 cm to 110 cm in width and 190 cm to 220 cm in length in some embodiments.

The following describes the first preset condition to the fourth preset condition in detail with specific embodiments. In this embodiment, the distance sensor array includes 6 distance sensors, the distance sensors measure different directions, the measurement angles of the distance sensors are shown in the FIG. 6. For ease of description, the distance sensors are respectively represented by T1-T6, and the bed size is 120 m×200 m. The first to fourth preset conditions are shown in Table 1.

Referring to FIG. 6, with the installation position as the origin and the length direction of the bed body as the reference direction, the measurement angle of Tl is offset to the right side of the bed by 17.83°; the measurement angle of T2 is offset to the left side of the bed by 6.48°; the measurement angle of T3 is offset to the left side of the bed by 18.53°; the measurement angle of T4 is offset to the left side of the bed by 39.36°; the measurement angle of T5 is offset to the left side of the bed by 52.18°; and the measurement angle of T6 is offset to the left side of the bed by 68.24°.

If the T2 reading and the T3 reading are both larger than 2 m, and the T1 reading and the T6 reading are both larger than 2 m, and the temperature difference between the thermal sensor reading and the temperature sensor reading is larger than 2 degrees Celsius, and usually the thermal sensor reading approximates the human body temperature (36 plus or minus 2 degrees Celsius), the patient is lying on the bed.

If the T2 reading or T3 reading is smaller than 2 m, and larger than 1.5 m, and the T1 or T6 reading is larger than 2 m, and the temperature difference between the thermal sensor reading and the temperature sensor reading is larger than 2 degrees Celsius, the patient is sitting in the middle of the bed. In this case, the thermal sensor reading usually approximates the human body temperature (36 plus or minus 2 degrees Celsius).

If the T2 reading and the T3 reading are both smaller than 1.5 m, the T1 reading and the T6 reading are both larger than 2 m, and the temperature difference between the thermal sensor reading and the temperature sensor reading is larger than 2 degrees Celsius, the patient is sitting on the right side of the bed. In this case, the thermal sensor reading usually approximates the human body temperature (36 plus or minus 2 degrees Celsius).

If both the T2 reading and the T3 reading are larger than 2 m, T4 reading or T5 reading or T6 reading is smaller than 1 m, and the temperature difference between the thermal sensor reading and the temperature sensor reading is larger than 2 degrees Celsius, the patient is sitting on the left side of the bed. In this case, the thermal sensor reading usually approximates the human body temperature (36 plus or minus 2 degrees Celsius).

If the T2 reading and T3 reading are larger than 2 m, and the T1 reading is smaller than 1.5 m, and the temperature difference between the thermal sensor reading and the temperature sensor reading is smaller than 2 degrees Celsius, the patient leaves the right side of the bed. In this case, the thermal sensor reading is approximately room temperature (around 25 degrees Celsius)

If the T2 and T3 and T4 and T5 readings are larger than 2 m, and the T6 reading is smaller than 1 m, and the temperature difference between the thermal sensor reading and the temperature sensor reading is smaller than 2 degrees Celsius, the patient leaves the left side of the bed. In this case, the thermal sensor reading is approximately room temperature (around 25 degrees Celsius).

If the T1 or T6 reading is smaller than 1.5 m and was previously in a standby state, a caregiver is present.

The first preset condition is that the patient is lying on the bed or sitting in the middle of the bed, which is safe when the first preset condition is met. The second preset condition is that the patient is sitting on the right or left side of the bed, which is abnormal when the second preset condition is met. The third preset condition is that the patient leaves the right or left side of the bed, which is in high risk when the third preset condition is met. The fourth preset condition is that a caregiver is present.

Optionally, the status of the patient can be determined comprehensively by combining the preset conditions and the previous state to reduce the error rate and improve the monitoring accuracy. For example, if the first preset condition is met and the previous state is standby, it is in the safe state; if the second preset condition is met and the previous state is safe, it is in the abnormal state; if the third preset condition is met and the previous state is abnormal, it is in the high risk state; if the fourth preset condition is met and the previous state is in high risk, a caregiver is present.

TABLE 1

| | Stand | | | | Nurse |
|---|---|---|---|---|---|
| | by | Safe | Abnormal | High risk | operation |
| Related to | n/a | Lying or Sitting, wherein | Sitting at right bed or left bed | Leave at right or left bedside. | (T1 or T6) reading < |
| distance sensor | | Lying-((T2 & T3) reading > | side. wherein, Sitting at right | Leave at right bedside-((T2 & T3) | 1.5m) & (Previous |
| | | 2m) & ((T1 & T6) | bed side.-((T2 & T3)reading < | reading > 2m) & (T1 reading < | state = Standby |
| | | reading > 2m) & (Previous | 1.5m) & (T1 & T6)reading > 2m)& | 1.5m) & (Previous state = | state) |
| | | state = standby state) | (Previous state = Safe state) | abnormal state) | |
| | | Sitting-(1.5m < (T2 or T3) | Sitting at left bed side.-((T2 & T3) | Leave at left bedside-((T2 & T3& | |
| | | reading < 2m) & ((T1 or T6) | reading > 2m) & (T4 or T5 Or T6) | T4 &T5) reading > 2m) & (T6 | |
| | | reading > 2m) & (Previous | reading < 1m) & (Previous state = | reading < 1.5m) & (Previous | |
| | | state = standby state) | safe state) | state = abnormal state) | |
| Related to | n/a | Thermal reading | Thermal reading approximates to | Thermal sensor reading | n/a |
| thermal sensor | | approximates to human | human body temperature(36 ± | approximates to temperature | |
| | | body temperature(36 ± | 2 degree Celsius) | sensor reading(36 ± 2 | |
| | | 2 degree Celsius) | | degree Celsius) | |

In an embodiment, when the position state of the human body is an abnormal or high-risk state, the speaker emits a corresponding sound prompt.

Table 2 shows one possible form of remote device alarm, when the human body position state is the safe state, the remote device sends out a green prompt without a sound alarm; when the human body position state is the abnormal state, the remote device sends out a yellow prompt without a sound alarm; and when the human body position state is the high-risk state, the remote device sends out a red prompt and sends out a sound alarm. In the system standby state, the remote device does not make a sound prompt and displays gray. When it is determined that a caregiver is present around the bed, the remote device displays grey, and no audible alarm.

TABLE 2

| | Standby | Safe | Abnormal | High risk | Nurse operation |
|---|---|---|---|---|---|
| action | Pause by nurses for | Lying and siting at | Sitting at bed side | Leave bed | Presence of Care |
| | long time bed transfer | center part of bed | | | |
| | action in hospital | | | | |
| Remote Device | Grey in dashboard | Green in dashboard | Yellow in dashboard | Red in dashboard | Grey in dashboard |
| Visual Response | | | | | |
| Remote device | No sound | No sound | No sound | Sound out | No sound |
| auditory response | | | | | |
| Speaker | No sound | No sound | Sending corresponding | Sending corresponding | No sound |
| | | | sound prompts | sound prompts | |

The illustrations of embodiments of the invention described herein are intended to provide a general understanding of the various embodiments, and they are not intended to serve as a complete description of all the elements and features of apparatus and systems that might make use of the circuits and techniques described herein. Many other embodiments will become apparent to those skilled in the art given the teachings herein; other embodiments are utilized and derived therefrom, such that structural and logical substitutions and changes can be made without departing from the scope of this disclosure. The drawings are also merely representational and are not drawn to scale. Accordingly, the specification and drawings are to be regarded in an illustrative rather than a restrictive sense.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. Terms such as “above,” “below,” “upper” and “lower” are used to indicate a position of elements or structures relative to one another, rather than absolute positioning.

The corresponding structures, materials, acts, and equivalents of all means or step-plus-function elements in the claims below are intended to include any structure, material, or act for performing the function in combination with other claimed elements as specifically claimed. The description of the various embodiments has been presented for purposes of illustration and description, but is not intended to be exhaustive or limited to the forms disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand the various embodiments with various modifications as are suited to the particular use contemplated.

What is claimed is:

1. A bed exit monitoring system comprising:
a distance sensor array comprising distance sensors and a sensor array module, each distance sensor operable to measure the distance between the human and the sensor in different angles, and output corresponding distance signal; and the sensor array module operable to receive and process the distance signals output by each distance sensor and output a distance signal matrix according to all the distance signals;
a thermal sensor configured to measure a temperature in a range of the bed and output a corresponding temperature signal;
a processing unit, configured to process a distance signal matrix of the distance sensor array and a temperature signal of the thermal sensor to determine a human body position, and output a position state signal of the human body;
a first alarm unit, configured to identify the position state signal output by the processing unit and make corresponding feedback.

2. The bed exit monitoring system according to claim 1, wherein the detection angles of the distance sensors are different, and the detection angles of the distance sensor array covers the whole bed body and the vicinity of the bed.

3. The bed exit monitoring system according to claim 1, wherein the distance sensor is a ToF lidar sensor.

4. The bed exit monitoring system according to claim 1, further comprising a temperature sensor, wherein the temperature sensor communicates with the processing unit, and is configured to measure a room temperature and output a corresponding temperature signal.

5. The bed exit monitoring system according to claim 4, wherein a classification module is arranged in the processing unit, and programmed to classify a human body position into safe, abnormal, or high-risk states based on the distance signal matrix of the distance sensor array and the temperature signal output by the thermal sensor and the temperature sensor.

6. The bed exit monitoring system according to claim 1, further comprising a remote device, wherein the remote device comprises a second alarm unit, the second alarm unit is configured to identify the position state signal output by the processing unit and make a corresponding feedback, and the second alarm unit generates an auditory alarm and/or a visual alarm.

7. The bed exit monitoring system according to claim 6, wherein the remote device further comprises a display unit, and the display unit communicates with the processing unit and is configured to present a real-time human body position states.

8. The bed exit monitoring system according to claim 1, further comprising a communication module configured to transmit a position state signal of the human body.

9. The bed exit monitoring system according to claim 6, further comprising a cloud server, wherein the processing unit uploads the position state signal to the cloud server, and the cloud server sends the position state signal to the remote device.

10. A bed exit monitoring device comprising: a case, a distance sensor array, a thermal sensor, a print circuit board assembly, and a speaker assembly; wherein the case is provided with a chamber set inside, and a switch set outside;
the distance sensor array, disposed on the case and face the bed body, comprises distance sensors and a sensor array module;
the thermal sensor is disposed on the case and faces the bed body;
the circuit board assembly is disposed in the chamber of the case and comprises a processor, and the processor is in communication with the distance sensor array and the thermal sensor;
the speaker assembly is disposed on the case and in communication with the processor.

11. The bed exit monitoring device according to claim 10, further comprising a bracket disposed on the bed frame and configured for the case installation.

12. The bed exit monitoring device according to claim 10, wherein the case is configured as a shape of a cat, and comprises a front case and a rear case, and the front case and the rear case are detachably connected; the speaker assembly comprises a speaker and a volume button, the speaker is disposed on the front case, and the volume button is disposed on the rear case.

13. The bed exit monitoring device according to claim 10, further comprising a temperature sensor disposed on the case and configured to measure the room temperature.

14. The bed exit monitoring device according to claim 10, further comprising a remote device, wherein the remote device is in communication with the processor.

15. The bed exit monitoring device according to claim 14, further comprising a cloud server, wherein the cloud server is in communication connection with the processor and the remote device respectively.

16. An bed exit monitoring method comprising:
installing the bed exit monitoring device on the bed frame;
witching on the device and connecting to a cloud server;
measuring, by the distance sensor array, the distance between human body and the distance sensor array; and measuring, by the thermal sensor, the temperature within the bed body; and measuring, by the temperature sensor, the room temperature;
determining, by the processor, a human body position based on the spatial distance readings from the sensor array and also the temperature difference between the thermal sensor reading and the temperature sensor reading, and determining which human body position state the human in;
uploading, by the processor, the human body position states to a cloud server;
sending, by the cloud server, the human body position states to the remote device;
displaying, by the remote device, a human body position state, and feeding back corresponding prompt information in different states.

17. The bed exit monitoring method according to claim 16, wherein the step of measuring comprises: each distance sensor in the distance sensor array measures a distance between a human body and a sensor in different angles, and outputs a corresponding distance signal; a sensor array module of the distance sensor array receives and processes the distance signal output by each distance sensor, and outputs a distance signal matrix according to all the distance signals.

18. The bed exit monitoring method according to claim 17, wherein the distance sensor array's measurement range further comprises the area around the bed.

19. The bed exit monitoring method according to claim 16, wherein the determining which human body position state the human in comprises:
when the results of the distance sensor array readings and the temperature difference between the thermal sensor reading and the temperature sensor reading meet a first preset condition, determining, by the processor, the position of the human body as a safe state;
when the above results meet a second preset condition, determining, by the processor, the human body position as an abnormal state;
when the above results meet a third preset condition, determining, by the processor, the human body position as a high-risk state;
when the above results meet the fourth preset condition, determining, by the processor, that a caregiver exists around the bed body.

20. The method according to claim 19, wherein the first preset condition to the fourth preset condition are set according to a spatial size of the detection range.
